(19) United States
(12) Patent Application Publication
(10) Pub. No.: US 2004/0019018 A1
(43) Pub. Date: Jan. 29, 2004

(54) Rigid pyrrolidone modulators of pkc

(76) Inventors: Lixin Qlao, Tewksbury, MA (US); Alan Kozikowski, Princeton, NJ (US); Lianyun Zhao, Lincoln, RI (US); Rene Etcheberrigaray, Columbia, MD (US)

Correspondence Address: VENABLE, BAETJER, HOWARD AND CIVILETTI, LLP, P.O. BOX 34385, WASHINGTON, DC, 20043-9998, US

(21) Appl. No.: 10/258,661

(22) PCT Filed: Apr. 30, 2001
(86) PCT No.: PCT/US01/13667

(51) Int. Cl. A61K 31/675; C07F 9/547; A61K 31/403

(52) U.S. Cl. 514/80; 514/411; 548/414; 548/431

(57) ABSTRACT

Compounds of the general formula (I): wherein substituents at R¹, R², R³ and R⁴ have any of the values defined in the specification, and their pharmaceutically acceptable salts, are PKC modulators and are useful for treating diseases, such as, for example, cancers including prostate cancer, inflammatory, autoimmune and neurological disorders including Alzheimer's disease. Also disclosed are pharmaceutical compositions comprising compounds of formula (I), processes for preparing compounds of formula (I), and intermediates useful for preparing compounds of formula (I).

### GOVERNMENT SUPPORT

This invention was made with U.S. Government support under CA79601 awarded by the National Institutes of Health. The Government may have certain rights in the invention.

### FIELD OF THE INVENTION

This invention relates to molecules that modulate the biological activities of isozymes of protein kinase C (PKC), thereby affecting diverse cellular functions, including cell growth, cell differentiation, apoptosis, ion channel activity, neurotransmitter release, and neuronal plasticity. In particular, the invention relates to tricyclic, rigid pyrrolidone compounds and derivatives thereof that bind to PKC, comprise a tricyclic structure of fused pyrrole, cycloalkyl, and phenyl rings, and mimic the binding of naturally occurring modulators of PKC activity such as diacylglycerol and indolactam V (ILV). The compounds of the present invention may be advantageously used, for example, in the treatment of cancerous, autoimmune, inflammatory and neurologic diseases, and for the treatment of conditions associated with amyloid processing and plaque formation.

### BACKGROUND OF THE INVENTION

Protein kinases serve diverse regulatory functions which are crucial for all aspects of cellular development, differentiation, and transformation. One of the largest gene families of non-receptor serine-threonine protein kinases is protein kinase C (PKC). Since the discovery of PKC more than a decade ago by Nishizuka and coworkers (Kikkawa et al., J. Biol. Chem., 257, 13341 (1982)), and its identification as a major receptor for phorbol esters (Ashendel, et al., Cancer Res., 43, 4333 (1983)), a multitude of physiological signaling mechanisms have been ascribed to this enzyme. The intense interest in PKC stems from its unique ability to be activated in vitro by diacylglycerol (and its phorbol ester mimetics), an effector whose formation is coupled to phospholipid turnover by the action of growth and differentiation factors.

The PKC gene family consists presently of 11 genes which are divided into four subgroups: 1) classical PKCα, β₁, β₂ (β₁ and β₂ are alternately spliced forms of the same gene) and γ; 2) novel PKC delta (δ), epsilon (ε), eta (η), and theta (θ); 3) atypical PKC zeta (ζ), lambda (λ) and iota (ι); and 4) PKC μ. PKC μ resembles the novel PKC isozymes but differs by having a putative transmembrane domain (reviewed in Blobe, et al., Cancer Metast. Rev., 13, 411 (1994)). The α, β₁, β₂ and γ isozymes are Ca²⁺, phospholipid- and diacylglycerol-dependent and represent the classical isozymes of PKC, whereas the other isozymes are activated by phospholipid and diacylglycerol but are not dependent on Ca²⁺. All isozymes encompass 5 variable (V1-V5) regions, and the alpha, beta and gamma isozymes contain four (C1-C4) structural domains which are highly conserved. All isozymes except PKC alpha, beta, and gamma lack the C2 domain, and the lambda, eta and iota isozymes also lack one of two cysteine-rich zinc finger domains in C1 to which diacylglycerol binds. The C1 domain also contains the pseudosubstrate sequence which is highly conserved among all isozymes, and which serves an autoregulatory function by blocking the substrate-binding site to produce an inactive conformation of the enzyme (House et al. Science, 238, 1726 (1987)).

Because of these structural features, the various PKC isozymes are thought to have highly specialized roles in signal transduction (Nishizuka, Cancer, 10, 1892 (1989)), as well as in neoplastic transformation and differentiation (Glazer, Protein Kinase C, J. F. Kuo, ed., Oxford U. Press (1994) at pages 171-198.

From a pharmacological perspective, PKC has served as a focal point for the design of anticancer drugs (Gescher, Brit. J. Cancer, 66, 10 (1992)). Antisense expression of either the PKC α cDNA (Ahmad, et al., Neurosurgery, 35, 904 (1994)) or a phosphorothioate oligodeoxynucleotide (S-oligo) for PKC alpha has shown the efficacy of targeting PKC to inhibit the proliferation of A549 lung carcinoma cells (Dean, et al., J. Biol. Chem., 269, 16416 (1994)) and U-87 glioblastoma cells. However, it is not clear which isozymes are most crucial for tumor proliferation and what specific roles different PKC isozymes play in critical cellular processes such as cell proliferation and apoptosis.

Prostate cancer is a leading cause of cancer death among men in Western countries. Studies indicate that androgen-sensitive (comparatively benign) and androgen-insensitive (malignant) forms of the disease differ in their responses to PKC inhibitors. It has been found that the PKC activator 12-O-tetradecanoyl-phorbol-13-acetate (TPA) promotes cell death in androgen-sensitive LNCaP cells, rather than androgen-independent DU-145 or PC-3 cells, whose growth is significantly decreased by PKC inhibitor staurosporine. However, the effect of another PKC activator, bryostatin 1, on LNCaP cell viability could be reversed by altering the amount of overexpressing PKC α or ε (Goekjian, et al., Curr. Med. Chem. 6:877-903 (1999)). PKC α is therefore an attractive and novel target for the therapy of androgen-independent prostate cancer, because PKC isozymes are involved in the regulation of prostate cancer cell growth and apoptosis.

Investigations with TPA have provided considerable information on tumor promotion. In the presently accepted two stage model of skin carcinogenesis, initiators bind to DNA in a first step, and, in a second step, tumor promoters such as TPA bind non-covalently to membrane-associated high affinity receptors, possibly PKC. It is likely that TPA, as well as the known teleocidins, lyngbyatoxins, and aplysiatoxin, serve as diacylglycerol mimics in binding to the diacylglycerol site of PKC, thus activating the kinase.

There is a continuing need for novel compounds which can activate PKC, and, in particular, there is a need for compounds that selectively activate the different isozymes of PKC. Such compounds may be useful, for example, to effect the selective killing of cancer cells, and in the treatment of autoimmune, inflammatory and neurological diseases.

### SUMMARY OF THE INVENTION

The present invention provides certain substituted polycylic, rigid pyrrolidones that are PKC modulators. Accordingly, the invention provides a compound of the general structural formula (I):

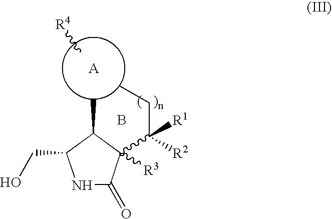

The compounds comprise a pyrrole ring fused to a first cyclic substituent (B), wherein the first cyclic substituent is also fused to a second cyclic substituent (A). The second cyclic substituent is preferably a carbocyclic ring, preferably a C₅-C₇ cycloalkyl group and more preferably a phenyl ring. The second cyclic substituent is may be substituted with one or more groups R⁴ In the compounds, R¹, R², and R³, are each independently hydrogen, (C₁-C₁₅)alkyl, (C₂-C₁₅)alkenyl, (C₂-C₁₅)alkynyl, (C₃-C₈)cycloalkyl, (C₃-C₈)cycloalkyl(C₁-C₁₀)alkyl, (C₃-C₈)cycloalkyl(C₁-C₁₅)alkenyl, (C₃-C₈)cycloalkyl(C₁-C₁₅)alkynyl, (C₁-C₁₅)alkoxy, (C₁-C₁₅)alkanoyl, (C₁-C₁₅)alkanoyloxy, aryl heteroaryl, aryl(C₁-C₆)alkyl, heteroaryl(C₁-C₆)alkyl, aryl(C₂-C₁₅)alkenyl, heteroaryl(C₂-C₁₅)alkenyl, aryl(C₂-C₁₅)alkynyl, heteroaryl(C₂-C₁₅)alkynyl, aryl(C₁-C₁₅)alkoxy, heteroaryl(C₁-C₁₅)alkoxy, aryl(C₁-C₁₅)alkanoyl, heteroaryl(C₁-C₁₅)alkanoyl, aryl(C₁-C₁₅)alkanoyloxy, or heteroaryl(C₁-C₁₅)alkanoyloxy; or R¹ and R² together form a cyclopropyl, cyclobutyl or cyclopentyl ring optionally substituted with one or more substituents R⁵ ring spirocyclic to said substituted cycloalkyl ring B; wherein R⁵ comprises one or more substituents independently selected from the group consisting of hydrogen, (C₁-C₁₅)alkyl, (C₂-C₁₅)alkenyl, (C₂-C₁₅)alkynyl, (C₃-C₈)cycloalkyl, (C₃-C₈)cycloalkyl(C₁-C₁₀)alkyl, (C₃-C₈)cycloalkyl(C₁-C₁₅)alkenyl, (C₃-C₈)cycloalkyl(C₁-C₁₅)alkynyl, (C₁-C₁₅)alkoxy, (C₁-C₁₅)alkanoyl, (C₁-C₁₅)alkanoyloxy, aryl heteroaryl, aryl(C₁-C₆)alkyl, heteroaryl(C₁-C₆)alkyl, aryl(C₂-C₁₅)alkenyl, heteroaryl(C₂-C₁₅)alkenyl, aryl(C₂-C₁₅)alkynyl, heteroaryl(C₂-C₁₅)alkynyl, aryl(C₁-C₁₅)alkoxy, heteroaryl(C₁-C₁₅)alkoxy, aryl(C₁-C₁₅)alkanoyl, heteroaryl(C₁-C₁₅)alkanoyl, aryl(C₁-C₁₅)alkanoyloxy, or heteroaryl(C₁-C₁₅)alkanoyloxy; and R³ is as defined above.

R⁴ comprises 0-4 substituents, independently selected from the group consisting of halo, nitro, cyano, hydroxy, phospho, sulfo, trifluoromethyl, trifluoromethoxy, (C₁-C₁₅)alkyl, (C₂-C₁₅)alkenyl, (C₂-C₁₅)alkynyl, (C₃-C₈)cycloalkyl, (C₃-C₈)cycloalkyl(C₁-C₁₅)alkyl, (C₃-C₈)cycloalkyl(C₂-C₁₅)alkenyl, (C₃-C₈)cycloalkyl(C₂-C₁₅)alkynyl, (C₁-C₁₅)alkoxy, (C₁-C₁₅)alkanoyl, (C₁-C₁₅)alkanoyloxy, C(═O)ORₐ, C(═O)NR_{b}R_{c}, OC(═O)ORₐ, OC(═O)NR_{b}R_{c}, and NR_{d}Rₑ; wherein each Rₐ is independently hydrogen or (C₁-C₆)alkyl; wherein each R_{b} and R_{c} is independently hydrogen or (C₁-C₁₀)alkyl; or R_{b}, and R_{c} together with the nitrogen to which they are attached are a 5-6 membered heterocyclic ring; and wherein each R_{d} and Rₑ is independently hydrogen, (C₁-C₁₀)alkyl, (C₁-C₁₀)alkanoyl, phenyl, benzyl, or phenethyl; or R_{d} and Rₑ together with the nitrogen to which they are attached are a 5-6 membered heterocyclic ring; n is 0, 1, or 2.

Preferred embodiments include compounds of Formula I and II, with the substituents as described above.

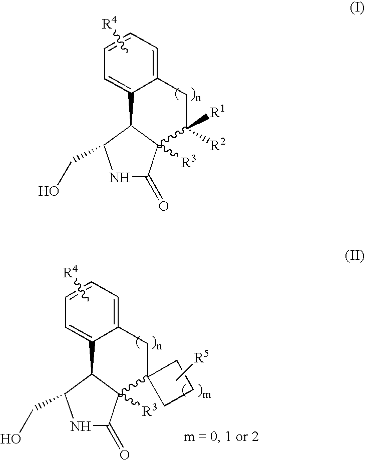

Pharmaceutically acceptable salts of the above compounds are included within the scope of the present invention, as are formulations of substituted tricyclic, rigid pyrrolidones with pharmaceutically acceptable carriers.

The invention also provides pharmaceutical compositions comprising a compound of the invention, processes for preparing compounds of the invention, and novel intermediates useful for the synthesis of compounds of the invention.

The invention also provides a therapeutic method comprising treating a condition characterized by the pathological proliferation of mammalian cells (e.g. cancers and tumors, and in a preferred embodiment, prostate cancer) by administering to a mammal afflicted with such a condition an effective amount of a substituted tricyclic, rigid pyrrolidone with a pharmaceutically acceptable carrier.

The invention also provides a method comprising modulating PKC activity in a mammal by administering to said mammal an effective dose of a substituted tricyclic, rigid pyrrolidone with a pharmaceutically acceptable carrier.

In a further embodiment, the invention provides a method of treating a patient having an autoimmune disease, by administering to said mammal an effective dose of a substituted tricyclic, rigid pyrrolidone with a pharmaceutically acceptable carrier.

In still a further embodiment, the invention provides a method of treating a patient having an inflammation, by administering to said mammal an effective dose of a substituted tricyclic, rigid pyrrolidone with a pharmaceutically acceptable carrier.

The present invention also relates to the use of rigid pyrrolidones to alter conditions associated with amyloid processing in order to enhance an α-secretase pathway to generate soluble α-amyloid precursor protein (α-APP) so as to reduce or prevent β-amyloid aggregation including β-amyloid accumulation in neurons. Such activation, for example, can be employed in the treatment of Alzheimer's disease, neurological disease, senile plaques, or cerebral amyloid angiopathy (CAA).

The present invention relates to a method for treating plaque formation, such as that associated with Alzheimer's disease, senile plaques, or CAA comprising administering to a subject an effective amount of a rigid pyrrolidone.

The invention is also a method of modulating K⁺ channel conductance by administering an effective amount of a pharmaceutical composition comprising a rigid pyrrolidone.

In another aspect, the present invention relates to a method for altering conditions associated with amyloid processing in order to enhance an α-secretase pathway to generate soluble α-amyloid precursor protein (α-APP) so as to prevent β-amyloid aggregation comprising administering a biologically effective amount of a rigid pyrrolidone.

In yet another aspect, the present invention relates to a composition for treating plaque formation, such as that associated with Alzheimer's disease comprising: a rigid pyrrolidone in an amount effective to generate soluble α-APP and reduce or prevent β-amyloid aggregation; and a pharmaceutically acceptable carrier.

It is reasonable to conclude that isozyme selective, non-tumor promoting activators of PKC may find use in cancer treatment through the initiation of cancer cell death by apoptosis. Selective cancer cell killing may be achieved either through the targeting of those isozymes found to be overexpressed in the cancer cells, or through the synergistic interaction of a cytotoxic drug like 1-beta-D-arabinofuranosylcytosine with an appropriate PKC-based signaling interceptor.

Because of their ability to modulate PKC, compounds of the invention may also be useful as pharmacological tools for the in vitro or in vivo study of the physiological function and effects of the PKC gene family.

The substituted tricyclic, rigid pyrrolidone compounds of the present invention exhibit distinct advantages over the prior art. These compounds have merit for several reasons, including availability, diversity, inexpensive starting material, and simplicity of the overall synthesis.

### BRIEF DESCRIPTION OF THE FIGURES

FIG. 1 illustrates the overall features of the binding model of a compound of the invention and a benzolactam in complex with PKCδ C1B.

FIG. 2 illustrates a synthetic reaction pathway for synthesis of a substituted tricyclic, rigid pyrrolidone of the present invention, (3S, 8R, 9S, 10S)-6-(dec-1′-ynyl)-3-hydroxymethyl-8-isopropyl-8-methyl-3,3a,8,8-tetrahydro-ZH-aza-cyclopenta[a]inden-1-one.

FIG. 3 illustrates synthetic reaction pathways for some di- and tri-substituted compounds of the invention.

FIG. 4 is a schematic representation of the various stages in the calcium signaling cascade.

FIG. 5 shows a western blot demonstrating the effect of compound 1 of the invention on sAPP α.

FIG. 6 is a graph of the induction of apoptosis in LNCaP prostate cancer cells by compound (1) or PMA.

### DETAILED DESCRIPTION OF THE INVENTION

SAR Study

In 1995, the X-ray crystal structures of PKCδ C1B in complex with phorbol 13-acetate revealed precisely how phorbol ester binds to PKC (Zhang, G.; Kazanietz, M. G.; Blumberg, P. M.; Hurley, J. H. Cell 1995, 81, 917) and provided a solid structural basis for the rational design of new PKC modulators (Wang, S.; Liu, M.; Lewin, N. E.; Lorenzo, P. S.; Bhattacharrya, d.; Qiao, L.; Kozikowski, A. P.; Milne, g. W. A.; Blumberg, P. M. J. Med. Chem. 1999, 42, 3436 and Kozikowski, A. P.; Wang, S.; Ma, D.; Yao, J.; Ahmad, S.; Glazer, R. I.; Bogi, I.; Acs, P.; Modarris, S.; Blumberg, P. M. J. Med. Chem. 1997, 40, 1316). Based upon the structure of the potent PKC activator 8-decynylbenzolactam (BL), and with the aid of molecular modeling, a series of pyrrolidone analogues (Formula I) were designed as a new class of PKC modulators (reference 11). These pyrrolidone analogues maintain the hydrogen bonding network and hydrophobic interactions considered crucial for interaction with PKC, and were found to possess good affinity for PKC and some isozyme selectivity. They avoid rotational flexibility of the aromatic ring.

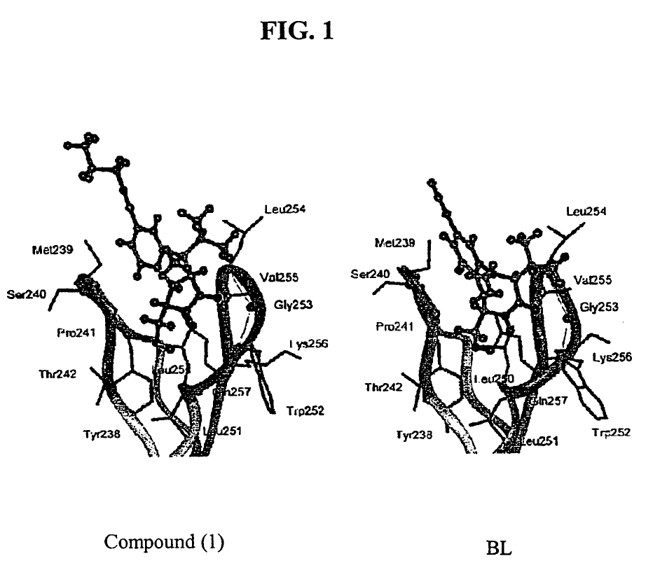

The N-1 atom of BL was replaced by a carbon atom, followed by shifting the isopropyl group from the previous C-2 position to the new carbon. C-2 and C-6 were subsequently joined to give the pyrrolidone system. Finally, the stereochemistry of all chiral centers was adjusted based upon results derived from molecular modeling to arrive at the structure of pyrrolidone (1). After energy minimization, the resulting conformation of compound (1) was superimposed upon BL. As depicted in FIG. 1, the crucial hydroxyl group and amide residue are preserved in compound (1), and these groups engage in a strong hydrogen bonding network with PKCδ C1B. The front methyl group on the marked chiral center mimics the N-Me in BL, interacting with Leu250 of PKC. Similarly, the rear isopropyl group of compound (1) interacts with side chain of Leu254, thereby mimicking the isopropyl group of BL. The same orientation of the phenyl group in compound (1) allows for strong hydrophobic interactions with Pro241 of PKC.

Compounds of the Invention

The following definitions are used, unless otherwise described. Halo is fluoro, chloro, bromo, or iodo. Alkyl, alkoxy, etc. denote both straight and branched groups; but reference to an individual radical such as “propyl” embraces only the straight chain radical, a branched chain isomer such as “isopropyl” being specifically referred to. Aryl denotes a phenylradical or an ortho-fused bicyclic carbocyclic radical having about nine to ten ring atoms in which at least one ring is aromatic. Heteroaryl encompasses a radical attached via a ring carbon of a monocyclic aromatic ring containing five or six ring atoms consisting of carbon and one to four heteroatoms each selected from the group consisting of non-peroxide oxygen, sulfur, and N(Rₓ) wherein Rₓ is absent or is hydrogen, oxo, (C₁-C₄)alkyl, phenyl or benzyl, as well as a radical of an ortho-fused bicyclic heterocycle of about eight to ten ring atoms derived therefrom, particularly a benzo-derivative or one derived by fusing a propylene, trimethylene, or tetramethylene diradical thereto.

It will be appreciated by those skilled in the art that compounds of the invention having one or more chiral center(s) may exist in and be isolated in optically active and racemic forms. Some compounds may exhibit polymorphism. It is to be understood that the present invention encompasses any racemic, optically-active, polymorphic, or stereoisomeric form, or mixtures thereof, of a compound of the invention, which possess the useful properties described herein, it being well known in the art how to prepare optically active forms (for example, by resolution of the racemic form by recrystallization techniques, by synthesis, from optically-active starting materials, by chiral synthesis, or by chromatographic separation using a chiral stationary phase) and how to determine the ability of a compound to activate PKC using the tests described herein, or using other tests which are well known in the art. The preferred absolute configuration for compounds of the invention is that shown in formula I above.

The term “cyclic substituent” herein denotes any aryl, heteroaryl, aromatic, cycloalkyl, or other cyclic moiety containing at least one continuous, cyclic pathway of covalently bonded constituent atoms.

The term “fused” herein denotes a covalent connection between two cyclic substituents of a molecule such that at least two adjacent atoms are common to both ring structures, and no independent rotational degree of freedom exists between the two cyclic substituents.

The term “tricyclic, rigid pyrrolidone” herein denotes a pyrrolidone of Formula III comprising a pyrrole ring fused to a first cyclic substituent (B), wherein the first cyclic substituent is also fused to a second cyclic substituent (A).

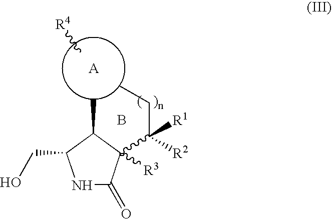

The second cyclic substituent is preferably a carbocyclic ring, preferably a C₅-C₇ cycloalkyl group and more preferably a phenyl ring. The second cyclic substituent is may be substituted with one or more groups R⁴. Compounds having a substituted or unsubstituted phenyl ring as the second cyclic substituent (Formula I) and a separate spirocyclic substituent as R¹ and R² (Formula II) are preferred tricyclic, rigid pyrrolidones.

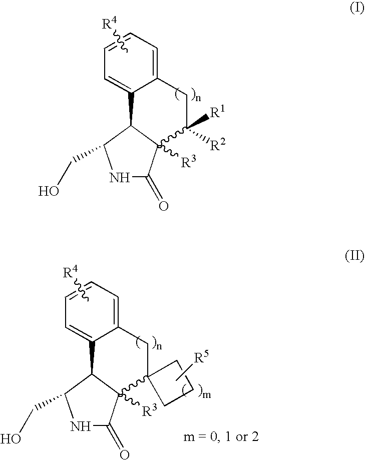

With respect to Formulas I to III, a specific value for R¹, R² or R³, is (C₁-C₁₅)alkyl, (C₂-C₁₅)alkenyl, C₂-C₁₅)alkynyl, (C₃-C₈) cycloalkyl, (C₃-C₈) cycloalkyl(C₁-C₁₀)alkyl, (C₃-C₈)cycloalkyl(C₁-C₁₅)alkenyl, (C₃-C₈)cycloalkyl(C₁-C₁₅)alkynyl, (C₁-C₁₅)alkoxy, (C₁-C₁₅)alkanoyl, or (C₁-C₁₅)alkanoyloxy; wherein R¹ is optionally substituted with one or more (e.g. 1, 2, 3, or 4) substituents independently selected from the group consisting of halo, nitro, cyano, hydroxy, trifluoromethyl, trifluoromethoxy, (C₁-C₁₅)alkyl, (C₂-C₁₅)alkenyl, (C₂-C₁₅)alkynyl, (C₃-C₈)cycloalkyl, (C₃-C₈)cycloalkyl (C₁-C₁₅)alkyl, (C₃-C₈)cycloalkyl-(C₂-C₁₅)alkenyl, (C₃-C₈)cycloalkyl(C₂-C₁₅)alkynyl, (C₁-C₁₅)alkoxy, (C₁-C₁₅)alkanoyl, (C₁-C₁₅)alkanoyloxy, C(═O)ORₐ, C(═O)NR_{b}R_{c}, OC(═O)ORₐ, OC(═O)NR_{b}R_{c}, and NRₑR_{f}.

Another specific value for R₁, R² or R³, is (C₁-C₁₅)alkyl, (C₂-C₁₅)alkenyl, (C₂-C₁₅)alkynyl, (C₃-C₈)cycloalkyl, (C₃-C₈)cycloalkyl(C₁-C₁₀)alkyl, (C₃-C₈)cycloalkyl(C₁-C₁₅)alkenyl, C₃-C₈)cycloalkyl(C₁-C₁₅)alkynyl, (C₁-C₁₅)alkoxy, (C₁-C₁₅)alkanoyl, or (C₁-C₁₅)alkanoyloxy; wherein R¹ is optionally substituted with one or more (e.g. 1, 2, 3, or 4) substituents independently selected from the group consisting of halo, nitro, cyano, hydroxy, trifluoromethyl, trifluoromethoxy, C(═O)ORₐ, C(═O)NR_{b}R_{c}, OC(═O)ORₐ, OC(═O)NR_{b}R_{c}, and NRₑR_{f}.

Another specific value for R¹, R² or R³, is aryl optionally substituted with 1, 2, or 3 substituents independently selected from the group consisting of halo, nitro, cyano, hydroxy, trifluoromethyl, trifluoromethoxy, (C₁-C₆)alkyl, (C₂-C₁₀)alkenyl, (C₂-C₁₀)alkynyl, (C₃-C₈)cycloalkyl, (C₃-C₈)cycloalkyl(C₁-C₆)alkyl, (C₁-C₁₀)alkoxy, (C₁-C₁₀)alkanoyl, (C₂-C₁₀)alkanoyloxy, C(═O)ORₐ, C(═O)NR_{b}R_{c}, or NRₑR_{f}.

Another specific value for R¹, R² or R³, is phenyl or naphthyl, optionally substituted with 1, 2, or 3 substituents independently selected from the group consisting of halo, nitro, cyano, hydroxy, trifluoromethyl, trifluoromethoxy, (C₁-C₆)alkyl, (C₂-C₁₀)alkenyl, (C₂-C₁₀) alkynyl, (C₃-C₈)cycloalkyl, (C₃-C₈)cycloalkyl(C₁-C₆)alkyl, (C₁-C₁₀)alkoxy, (C₁-C₁₀)alkanoyl, (C₂-C₁₀)alkanoyloxy, C(═O)ORₐ C(═O)NR_{b}R_{c}, or NRₑR_{f}.

Another specific value for R¹, R² or R³, is phenyl or naphthyl, optionally substituted with a substituent selected from the group consisting of halo, nitro, cyano, hydroxy, trifluoromethyl, trifluoromethoxy, (C₁-C₆)alkyl, (C₂-C₁₀)alkenyl, (C₂-C₁₀)alkynyl, (C₃-C₈)cycloalkyl, (C₃-C₈)cycloalkyl(C₁-C₆)alkyl, (C₁-C₁₀ alkoxy, (C₁-C₁₀)alkanoyl, (C₂-C₁₀)alkanoyloxy, C(═O)ORₐ, C(═O)NR_{b}R_{c}, or NRₑR_{f}.

Another specific value for R¹, R² or R³, is aryl, heteroaryl, aryl(C₁-C₆)alkyl, heteroaryl(C₁-C₆)alkyl, aryl(C₂-C₆)alkenyl, heteroaryl(C₂-C₆)alkenyl, aryl(C₂-C₆)alkynyl, or heteroaryl(C₂-C₆)alkynyl; wherein any aryl or heteroaryl of R¹ is optionally substituted with one or more (e.g. 1, 2, 3, or 4) substituents independently selected from the group consisting of halo, nitro, cyano, hydroxy, trifluoromethyl, trifluoromethoxy, (C₁-C₁₅)alkyl, (C₂-C₁₅)alkenyl, (C₂-C₁₅)alkynyl, (C₃-C₈)cycloalkyl, (C₃-C₈)cycloalkyl(C₁-C₁₅)alkyl, (C₃-C₈)cycloalkyl-(C₂-C₁₅)alkenyl, (C₃-C₈)cycloalkyl(C₂-C₁₅)alkynyl, (C₁-C₁₅)alkoxy, (C₁-C₁₅)alkanoyl, (C₁-C₁₅)alkanoyloxy, C(═O)ORₐ, C(═O)NR_{b}R_{c}, OC(═O)ORₐ, OC(═O)NR_{b}R_{c}, and NRₑR_{f}.

Another specific value for R¹, R² or R³, is aryl, heteroaryl, aryl(C₁-C₆)alkyl, heteroaryl(C₁-C₆)alkyl, aryl(C₂-C₆)alkenyl, heteroaryl(C₂-C₆)alkenyl, aryl(C₂-C₆)alkynyl, or heteroaryl(C₂-C₆)alkynyl; wherein any aryl or heteroaryl of R¹ is optionally substituted with halo, nitro, cyano, hydroxy, trifluoromethyl, trifluoromethoxy, (C₁-C₁₅)alkyl, (C₂-C₁₅)alkenyl, (C₂-C₁₅)alkynyl, (C₃-C₈)cycloalkyl, (C₃-C₈)cycloalkyl(C₁-C₁₅)alkyl, (C₃-C₈)cycloalkyl-(C₂-C₁₅)alkenyl, (C₃-C₈)cycloalkyl(C₂-C₁₅)alkynyl, (C₁-C₁₅)alkoxy, (C₁-C₁₅)alkanoyl, (C₁-C₁₅)alkanoyloxy, C(═O)ORₐ, C(═O)NR_{b}R_{c}, or NRₑR_{f}.

Another specific value for R¹, R² or R³, is aryl or heteroaryl wherein said aryl or heteroaryl is optionally substituted with one or more (e.g. 1, 2, 3, or 4) substituents independently selected from the group consisting of halo, nitro, cyano, hydroxy, trifluoromethyl, trifluoromethoxy, (C₁-C₁₅)alkyl, C₂-C₁₅)alkenyl, (C₂-C₁₅)alkynyl, (C₃-C₈)cycloalkyl, (C₃-C₈)cycloalkyl(C₁-C₁₅)alkyl, (C₃-C₈)cycloalkyl-(C₂-C₁₅)alkenyl, (C₃-C₈)cycloalkyl-(C₂-C₁₅)alkenyl, (C₃-C₈)cycloalkyl(C₂-C₁₅)alkynyl, (C₁-C₁₅)alkoxy, (C₁-C₁₅)alkanoyl, (C₁-C₁₅)alkanoyloxy, C(═O)ORₐ, C(═O)NR_{b}R_{c}, OC(═O)ORₐ, OC(═O)NR_{b}R_{c}, and NRₑR_{f}.

Another specific value for R¹, R² or R³, is aryl or heteroaryl, wherein said aryl or heteroaryl is optionally substituted with halo, nitro, cyano, hydroxy, trifluoromethyl, trifluoromethoxy, (C₁-C₁₅)alkyl, (C₂-C₁₅)alkenyl, (C₂-C₁₅)alkynyl, (C₃-C₈)cycloalkyl, (C₃-C₈)cycloalkyl(C₁-C₁₅)alkyl, (C₃-C₈)cycloalkyl-(C₂-C₁₅)alkenyl, (C₃-C₈)cycloalkyl(C₂-C₁₅)alkynyl, (C₁-C₁₅)alkoxy, (C₁-C₁₅)alkanoyl, (C₁-C₁₅)alkanoyloxy, C(═O)ORₐ, C(double bond O)NR_{b}R_{c}, OC(═O)OR₃, OC(=O) NR_{b}R_{c}, and NRₑR_{f}.

Another specific value for R¹, R² or R³, is aryl wherein said aryl is optionally substituted with one or more (e.g. 1, 2, 3, or 4) substituents independently selected from the group consisting of halo, nitro, cyano, hydroxy, trifluoromethyl, trifluoromethoxy, (C₁-C₁₅)alkyl, (C₂-C₁₅)alkenyl, (C₂-C₁₅)alkynyl, (C₃-C₈)cycloalkyl, (C₃-C₈)cycloalkyl(C₁-C₁₅)alkyl, (C₃-C₈)cycloalkyl-(C₂-C₁₅)alkenyl, (C₃-C₈)cycloalkyl(C₂-C₁₅)alkynyl, (C₁-C₁₅)alkoxy, (C₁-C₁₅)alkanoyl, (C₁-C₁₅)alkanoyloxy C(═O)ORₐ, C(═O)NR_{b}R_{c}, OC(═O)ORₐ, OC(═O)NR_{b}R_{c}, and NRₑR_{f}.

Another specific value for R¹, R² or R³, is aryl wherein said aryl is optionally substituted with halo, nitro, cyano, hydroxy, trifluoromethyl, trifluoromethoxy, (C₁-C₁₅)alkyl, (C₂-C₁₅)alkenyl, (C₂-C₁₅)alkynyl, (C₃-C₈)cycloalkyl, (C₃-C₈)cycloalkyl(C₁-C₁₅)alkyl, (C₃-C₈)cycloalkyl-(C₂-C₁₅)alkenyl, (C₃-C₈)cycloalkyl(C₂-C₁₅)alkynyl, (C₁-C₁₅)alkoxy, (C₁-C₁₅)alkanoyl, (C₁-C₁₅)alkanoyloxy, C(═O)ORₐ, C(═O)NR_{b}R_{c}, OC(═O)ORₐ, OC(═O)NR_{b}R_{c}, and NRₑR_{f}.

Another specific value for R¹, R² or R³, is aryl or heteroaryl wherein said aryl or heteroaryl is substituted with one or more (e.g. 1, 2, 3, or 4) substituents independently selected from the group consisting of halo, nitro, cyano, hydroxy, trifluoromethyl, trifluoromethoxy, (C₃-C₈)cycloalkyl, (C₃-C₈)cycloalkyl(C₁-C₁₅)alkyl, (C₃-C₈)cycloalkyl(C₂-C₁₅)alkenyl, (C₃-C₈)cycloalkyl(C₂-C₁₅)alkynyl, (C₁-C₁₅)alkoxy, (C₁-C₁₅)alkanoyl, (C₁-C₁₅)alkanoyloxy, C(═O)ORₐ, C(═O)NR_{b}R_{c}, OC(═O)ORₐ, OC(═O)NR_{b}R_{c}, and NRₑR_{f}.

Another specific value for R¹, R² or R³,is phenyl or naphthyl, wherein said phenyl or naphthyl is optionally substituted with one or more (e.g. 1, 2, 3, or 4) substituents independently selected from the group consisting of halo, nitro, cyano, hydroxy, trifluoromethyl, trifluoromethoxy, (C₁-C₆)alkyl, (C₂-C₆)alkenyl, (C₂-C₆)alkynyl, (C₃-C₈)cycloalkyl, (C₃-C₈)cycloalkyl(C₁-C₆)alkyl, (C₃-C₈)cycloalkyl(C₁-C₆)alkenyl, (C₃-C₈)cycloalkyl(C₁-C₆)alkynyl, (C₁-C₆)alkoxy, (C₁-C₆)alkanoyl, (C₂-C₆)alkanoyloxy, C(═O)ORₐ, C(═O)NR_{b}R_{c}, OC(═O)ORₐ, OC(═O)NR_{b}R_{c}, and NRₑR_{f}.

Another specific value for R¹, R² or R³, is phenyl or naphthyl, wherein said phenyl or naphthyl is optionally substituted with halo, nitro, cyano, hydroxy, trifluoromethyl, trifluoromethoxy, (C₁-C₁₅)alkyl, (C₂-C₁₅)alkenyl, (C₃-C₈)cycloalkyl(C₁-C₁₅)alkyl, (C₃-C₈)cycloalkyl-(C₂-C₁₅)alkenyl, (C₃-C₈)cycloalkyl(C₂-C₁₅)alkynyl, (C₁-C₁₅)alkoxy, (C₁-C₁₅)alkanoyl, (C₁-C₁₅)alkanoyloxy, C(═O)ORₐ, C(═O)NR_{b}R_{c}, OC(═O)ORₐ, OC(═O)NR_{b}R_{c}, and NRₑR_{f}.

Another specific value for R¹, R² or R³, is aryl or heteroaryl, wherein said aryl or heteroaryl is substituted with (C₁-C₁₅)alkyl, (C₂-C₁₅)alkenyl, (C₂-C₁₅)alkynyl, (C₁-C₁₅)alkoxy, (C₁-C₁₅)alkanoyl, or (C₁-C₁₅)alkanoyloxy, and can also be optionally substituted with 1 or 2 halo, nitro, cyano, hydroxy, trifluoromethyl, trifluoromethoxy, (C₁-C₆)alkyl, (C₃-C₈)cycloalkyl, (C₁-C₆)alkoxy, (C₁-C₆)alkanoyl, (C₂-C₆)alkanoyloxy, C(═O)ORₐ, C(═O)NR_{b}R_{c}, or NRₑR_{f}.

Another specific value for R¹ is phenyl or naphthyl, wherein said phenyl or naphthyl is substituted (preferably at the 4-position) with (C₁-C₁₅)alkyl, (C₂-C₁₅)alkenyl, (C₂-C₁₅)alkynyl (C₁-C₁₅)alkoxy, (C₁-C₁₅)alkanoyl, or (C₁-C₁₅)alkanoyloxy, and can also be optionally substituted with 1 or 2 halo, nitro, cyano, hydroxy, trifluoromethyl, trifluoromethoxy, (C₁-C₆)alkyl, (C₃-C₈)cycloalkyl, (C₁-C₆)alkoxy, (C₁-C₆)alkanoyl, (C₂-C₆)alkanoyloxy, C(═O)ORₐ, C(═O)NR_{b}R_{c}, or NRₑR_{f}.

A more specific value for R¹, R² or R³, is phenyl or naphthyl; optionally substituted with 1, 2, or 3 substituents independently selected from the group consisting of halo, nitro, cyano, hydroxy, trifluoromethyl, trifluoromethoxy, (C₁-C₆)alkyl, (C₂-C₁₀)alkenyl, (C₂-C₁₀)alkynyl(C₃-C₈)cycloalkyl, (C₃-C₈)cycloalkyl(C₁-C₆)alkyl, (C₁-C₁₀)alkoxy, C₁-C₁₀)alkanoyl, (C₂-C₁₀)alkanoyloxy, C(═O)ORₐ, C(═O)NR_{b}R_{c}, or NRₑR_{f}; Rₐ is hydrogen or (C₁-C₆)alkyl. Another more specific value for R¹, R² or R³, is phenyl or naphthyl, wherein said phenyl or naphthyl is substituted (preferably at the 4-position) with (C₁-C₁₅)alkyl, (C₂-C₁₅)alkenyl, (C₂-C₁₅)alkynyl (C₁-C₁₅)alkoxy, (C₁-C₁₅)alkanoyl, or (C₁-C₁₅)alkanoyloxy.

Another more specific value for R¹, R² or R³, is phenyl or naphthyl, wherein said phenyl or naphthyl is substituted (preferably at the 4-position with (C₁-C₁₅)alkyl, (C₂-C₁₅)alkenyl, or (C₂-C₁₅)alkynyl.

Another more specific value for R¹, R² or R³, is phenyl substituted (preferably at the 4-position) with (C₈-C₁₅)alkyl, (C₈-C₁₅)alkenyl, (C₈-C₁₅)alkynyl, (C₈-C₁₅)alkoxy, (C₈-C₁₅)alkanoyl, or (C₈-C₁₅)alkanoyloxy.

Another more specific value for R¹, R² or R³, is aryl (e.g. phenyl, naphthyl, or 5, 6, 7, 8-tetrahydronaphthyl) substituted (preferably at the 4-position) with (C₇-C₁₀)alkyl, (C₇-C₁₀) alkenyl, C₇-C₁₀)alkynyl, (C₇-C₁₀)alkoxy, (C₇-C₁₀)alkanoyl, or (C₇-C₁₀)alkanoyloxy.

Another more specific value for R¹ is naphthyl, optionally substituted (preferably at the 4-position) with (C₁-C₁₅)alkyl, (C₂-C₁₅)alkenyl, (C₂-C₁₅)alkynyl, (C₁-C₁₅)alkoxy, (C₁-C₁₅)alkanoyl, or (C₁-C₁₅)alkanoyloxy.

Another more specific value for R¹, R² or R³, is 5, 6, 7, 8-tetrahydronaphthyl, optionally substituted with (C₁-C₁₅)alkyl, (C₂-C₁₅)alkenyl, (C₂-C₁₅)alkynyl, (C₁-C₁₅)alkoxy, (C₁-C₁₅)alkanoyl, or (C₁-C₁₅)alkanoyloxy, and optionally substituted at the 5, 6, 7, or 8 position with a divalent (C₂-C₇)alkylene chain to form a (C₃-C₈)spirocycloalkyl.

A preferred value for R¹, R² or R³, is 4-nonylphenyl, phenyl, 1-naphthyl, 4-hexanoyloxynaphth-1-yl, 4-nonanoyloxynaphth-1-yl, 4-(1-hexynyl)naphth-1-yl, 7,7-dimethyl-4-nonanoyloxy-5,6,7,8-tetrahydronaphth-1-yl, 4-nonanoyloxy-5,6,7,8-tetrahydronaphth-1-yl, 4-hexanoyloxy-5,6,7,8tetrahydronaphth-1-yl, 4-nonanoyloxy-7-spirocyclopropyl-5,6,7,8-tetrahydronaphth-1-yl, or 3-pentyl.

A specific value for R¹, R² or R³, is (C₁-C₁₀)alkyl, (C₂-C₁₀)alkenyl, (C₃-C₈)cycloalkyl, (C₃-C₈)cycloalkyl(C₁-C₆)alkyl, (C₁-C₁₀)alkoxy, (C₁-C₁₀)alkanoyl, or (C₁-C₁₀)alkanoyloxy, optionally substituted with 1, 2, or 3 substituents independently selected from the group consisting of halo, nitro, cyano, hydroxy, trifluoromethyl, trifluoromethoxy, (C₁-C₆)alkyl, (C₂-C₁₀)alkenyl, (C₂-C₁₀)alkynyl, (C₃-C₈)cycloalkyl, (C₃-C₈)cycloalkyl(C₁-C₆)alkyl, (C₁-C₁₀) alkoxy, (C₁-C₁₀)alkanoyl, (C₂-C₁₀)alkanoyloxy, C(═O)ORₐ, C(═O)NR_{b}R_{c}, or NRₑR_{f}.

Another specific value for R¹, R² or R³, is (C₁-C₁₀)alkyl, (C₂-C₁₀)alkenyl, (C₃-C₈)cycloalkyl, (C₃-C₈)cycloalkyl(C₁-C₆)alkyl, (C₁-C₁₀)alkoxy, (C₁-C₁₀)alkanoyl, or (C₁-C₁₀)alkanoyloxy, optionally substituted with a substituent selected from the group consisting of halo, nitro, cyano, hydroxy, trifluoromethyl, trifluoromethoxy, (C₁-C₆)alkyl, (C₂-C₁₀)alkenyl, (C₂-C₁₀)alkynyl, (C₃-C₈)cycloalkyl, (C₃-C₈)cycloalkyl(C₁-C₆)alkyl, (C₁-C₁₀)alkoxy, (C₁-C₁₀)alkanoyl, (C₂-C₁₀)alkanoyloxy, C(═O)ORₐ, C(═O)NR_{b}R_{c}, or NRₑR_{f}.

Another specific value for R¹, R² or R³, is (C₁-C₁₀)alkyl, optionally substituted with 1, 2, or 3 substituents independently selected from the group consisting of halo, nitro, cyano, hydroxy, trifluoromethyl, trifluoromethoxy, (C₁-C₆)alkyl, (C₂-C₁₀)alkenyl, (C₂-C₁₀)alkynyl, (C₃-C₈)cycloalkyl, (C₃-C₈)cycloalkyl(C₁-C₆)alkyl, (C₁-C₁₀)alkoxy, (C₁-C₁₀)alkanoyl, (C₂-C₁₀)alkanoyloxy, C(═O)ORₐ, C(═O)NR_{b}R_{c}, or NRₑR_{f}.

Another specific value for R¹, R² or R³, is (C₁-C₁₀)alkyl, optionally substituted with a substituent selected from the group consisting of halo, nitro, cyano, hydroxy, trifluoromethyl, trifluoromethoxy, (C₁-C₆)alkyl, (C₂-C₁₀)alkenyl, (C₂-C₁₀)alkynyl, (C₃-C₈) cycloalkyl, (C₃-C₈)cycloalkyl(C₁-C₆)alkyl, (C₁-C₁₀)alkoxy, (C₁-C₁₀)alkanoyl, (C₂-C₁₀)alkanoyloxy, C(═O)ORₐ, C(═O)NR_{b}R_{c}, or NRₑR_{f}.

Another specific value for R¹, R² or R³, is (C₁-C₁₅)alkyl, (C₂-C₁₅)alkenyl, (C₂-C₁₅)alkynyl, (C₃-C₈) cycloalkyl, (C₃-C₈)cycloalkyl(C₁-C₁₀)alkyl, (C₁-C₁₅)alkoxy, (C₁-C₁₅)alkanoyl, (C₁-C₁₅)alkanoyloxy, aryl, heteroaryl, aryl(C₁-C₆)alkyl, heteroaryl (C₁-C₆)alkyl, aryl(C₂-C₁₅)alkenyl, heteroaryl(C₂-C₁₅)alkenyl, aryl(C₂-C₁₅)alkynyl, heteroaryl (C₂-C₁₅)alkynyl, aryl(C₁-C₁₅)alkoxy, heteroaryl(C₁-C₁₅)alkoxy, aryl(C₁-C₁₅)alkanoyl, heteroaryl(C₁-C₁₅)alkanoyl, aryl(C₁-C₁₅)alkanoyloxy, or heteroaryl(C₁-C₁₅)alkanoyloxy; wherein said R² is optionally substituted with one or more (e.g. 1, 2, 3, or 4) substituents independently selected from the group consisting of halo, nitro, cyano, hydroxy, trifluoromethyl, trifluoromethoxy, (C₁-C₆) alkoxy, (C₁-C₆)alkanoyl, (C₂-C₆)alkanoyloxy, C(═O) ORₐ, C(═O)NR_{b}R_{c}, and NRₑR_{f}.

Another specific value for R¹, R² or R³, is (C₁-C₁₅)alkyl, (C₂-C₁₅)alkenyl, (C₂-C₁₅)alkynyl, (C₃-C₈)cycloalkyl, (C₃-C₈)cycloalkyl(C₁-C₁₀)alkyl, (C₁-C₁₅)alkoxy, (C₁-C₁₅)alkanoyl, or (C₂-C₁₀)alkanoyloxy; wherein said R² is optionally substituted with one or more (e.g. 1, 2, 3, or 4) substituents independently selected from the group consisting of halo, nitro, cyano, hydroxy, trifluoromethyl, trifluoromethoxy, (C₁-C₆)alkoxy, (C₁-C₆)alkanoyl, (C₂-C₆)alkanoyloxy, C(═O)ORₐ, C(═O)NR_{b}R_{c}, and NRₑR_{f}.

Another specific value for R² is (C₁-C₁₅)alkyl, (C₂-C₁₅)alkenyl, or (C₂-C₁₅)alkynyl, wherein said R² is substituted with one or more (e.g. 1, 2, 3, or 4) substituents independently selected from the group consisting of halo, nitro, cyano, hydroxy, trifluoromethyl, trifluoromethoxy, (C₁-C₆)alkoxy, (C₁-C₆)alkanoyl, (C₂-C₆)alkanoyloxy, C(═O)ORₐ, C(═O)NR_{b}R_{c}, and NRₑR_{f}.

A more specific value for R² is (C₁-C₁₅)alkyl, (C₂-C₁₅)alkenyl, or (C₂-C₁₅)alkynyl.

Another more specific value for R² is (C₁-C₁₅)alkyl. Another more specific value for R² is (C₁-C₆)alkyl. Another more specific value for R² is (C₃-C₆)alkyl. A preferred value for R² is isopropyl or 3-pentyl.

Preferred Compounds

Processes for preparing compounds of Formula I are provided as further embodiments of the invention and are illustrated by the following procedures in which the meanings of the generic radicals are as given above unless otherwise qualified. The compounds of Formula I can be prepared from corresponding compounds, wherein the pyrrolidone nitrogen bears a suitable nitrogen protecting group, by deprotection of the nitrogen.

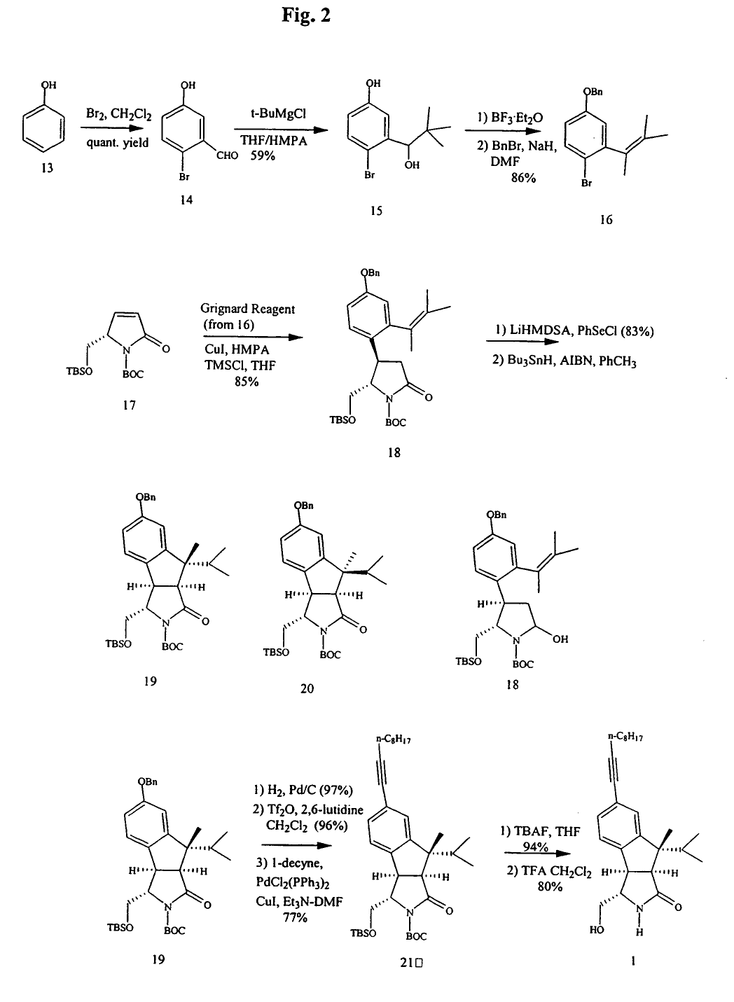

FIG. 2 illustrates the synthetic pathway for the synthesis of compound 1. For example, a compound of Formula I can be prepared by deprotection of a corresponding tert-butoxycarbonyl (BOC) protected pyrrolidone using conditions similar to those described in Example 1. Suitable nitrogen protecting groups are well known in the art (See Greene, T. W.; Wutz, P. G. M. “Protecting Groups In Organic Synthesis” second edition, 1991, New York, John Wiley & sons, Inc.).

Compounds of Formula I can be prepared from a corresponding compound wherein the hydroxymethyl of Formula I bears a suitable hydroxyl protecting group, by deprotection of the hydroxyl group. For example, a compound of Formula I can be prepared by deprotection of a corresponding tert-butyldimethylsilyl (TBS) protected alcohol using conditions similar to those described in Example 1. Suitable hydroxyl protecting groups are well known in the art (See Greene, T. W.; Wutz, P. G. M. “Protecting Groups In Organic Synthesis” second edition, 1991, New York, John Wiley & sons, Inc.).

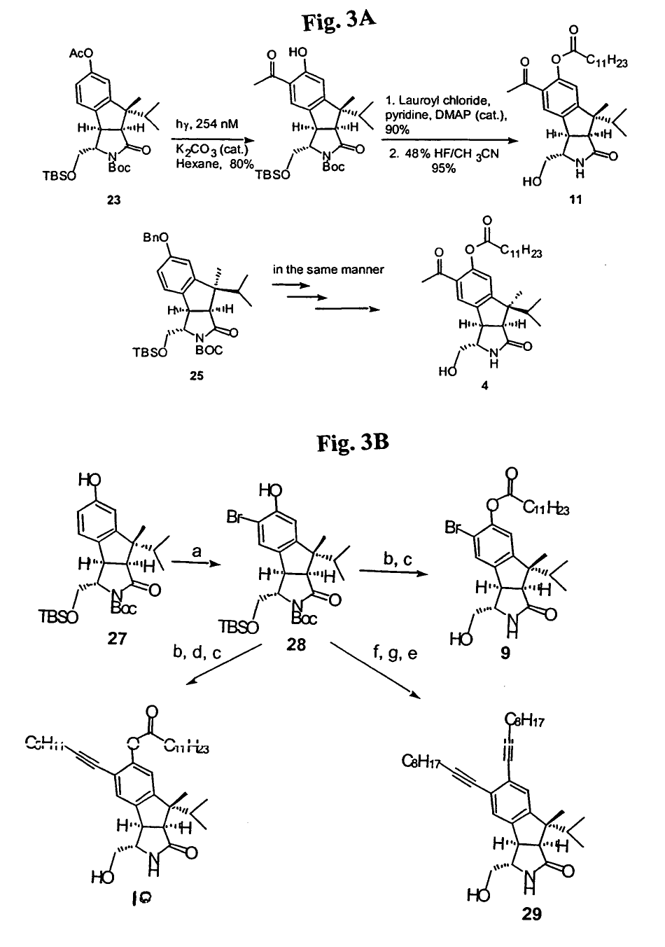

Compounds of the invention may also be prepared using procedures similar to those described in Examples 1 and 12, as illustrated in FIGS. 2-3.

In cases where compounds of Formula I are sufficiently basic or acidic to form stable nontoxic acid or base salts, administration of the compounds as salts may be appropriate. Examples of pharmaceutically acceptable salts are organic acid addition salts formed with acids which form a physiological acceptable anion, for example, tosylate, methanesulfonate, acetate, citrate, malonate, tartarate, succinate, benzoate, ascorbate, alpha-ketoglutarate, and alpha-glycerophosphate. Suitable inorganic salts may also be formed, including the hydrochloride salt and sulfate, nitrate, bicarbonate, and carbonate salts.

Pharmaceutically acceptable salts may be obtained using standard procedures well known in the art, for example by reacting a sufficiently basic compound such as an amine with a suitable acid affording a physiologically acceptable anion. Alkali metal (for example, sodium, potassium or lithium) or alkaline earth metal (for example calcium) salts of carboxylic acids can also be made.

The compounds of Formula I can be formulated as pharmaceutical compositions and administered to a mammalian host, such as a human patient, in a variety of forms adapted to the chosen route of administration, i.e., orally or parenterally, by intravenous, intramuscular, topical or subcutaneous routes.

Thus, the present compounds may be systemically administered, e.g., orally, in combination with a pharmaceutically acceptable vehicle such as an inert diluent or an assimilable edible carrier. They may be enclosed in hard or soft shell gelatin capsules, may be compressed into tablets, or may be incorporated directly with the food of the patient's diet. For oral therapeutic administration, the active compound may be combined with one or more excipients and used in the form of ingestible tablets, buccal tablets, troches, capsules, elixirs, suspensions, syrups, wafers, and the like. Such compositions and preparations should contain at least 0.1% of active compound. The percentage of the compositions and preparations may, of course, be varied and may conveniently be between about 2 to about 60% of the weight of a given unit dosage form. The amount of active compound in such therapeutically useful compositions is such that an effective dosage level will be obtained.

The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, cherry or orange flavoring. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active compound may be incorporated into sustained-release preparations and devices.

The active compound may also be administered intravenously or intraperitoneally by infusion or injection. Solutions of the active compound or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms suitable for injection or infusion can include sterile aqueous solutions or dispersions or sterile powders comprising the active ingredient which are adapted for the extemporaneous preparation of sterile injectable or infusible solutions or dispersions, optionally encapsulated in liposomes. In all cases, the ultimate dosage form must be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions are prepared by incorporating the active compound in the required amount in the appropriate solvent with various of the other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

For topical administration, the present compounds may be applied in pure form, i.e., when they are liquids. However, it will generally be desirable to administer them to the skin as compositions or formulations, in combination with a dermatologically acceptable carrier, which may be a solid or a liquid.

Useful solid carriers include finely divided solids such as talc, clay, microcrystalline cellulose, silica, alumina and the like. Useful liquid carriers include water, alcohols or glycols or water-alcohol/glycol blends, in which the present compounds can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user. Examples of useful dermatological compositions which can be used to deliver the compounds of formula I to the skin are disclosed in Jacquet et al. (U.S. Pat. No. 4,608,392), Geria (U.S. Pat. No. 4,992,478), Smith et al. (U.S. Pat. No. 4,559,157) and Wortzman (U.S. Pat. No. 4,820,508).

Useful dosages of the compounds of formula I can be determined by comparing their in vitro activity, and in vivo activity in animal models. Methods for the extrapolation of effective dosages in mice, and other animals, to humans are known to the art; for example, see U.S. Pat. No. 4,938,949.

Generally, the concentration of the compound(s) of formula I in a liquid composition, such as a lotion, will be from about 0.1-25 wt-%, preferably from about 0.5-10 wt-%. The concentration in a semi-solid or solid composition such as a gel or a powder will be about 0.1-5 wt-%, preferably about 0.5-2.5 wt-%. Single dosages for injection, infusion or ingestion will generally vary between 50-1500 mg, and may be administered, i.e., 1-3 times daily, to yield levels of about 0.5-50 mg/kg, for adults.

Accordingly, the invention includes a pharmaceutical composition comprising a compound of formula I as described above; or a pharmaceutically acceptable salt thereof, in combination with a pharmaceutically acceptable carrier. Pharmaceutical compositions adapted for oral, topical or parenteral administration, comprising an amount of one or more compounds of formula (I) effective to treat mammalian conditions associated with pathological cellular proliferation, particularly human cancers, such as solid tumors and leukemias, are a preferred embodiment of the invention.

Uses of the Compounds

The ability of a compound of the invention to activate PKC can be demonstrated using the pharmacological models described herein, or using other pharmacological models which are known in the art.

The X-ray crystal structures of PKC δ C1B in complex with phorbol 13-acetate reveal precisely how phorbol ester binds to PKC (Goekjian, et al., Curr. Med. Chem. 6:877-903 (1999)). The structure provides a solid basis for the rational design of new PKC modulators. The design concept of pyrrolidone derivatives as novel PKC modulators has been published in a previous paper (Qiao, et al., J. Am. Chem. Soc. 120:6629-6630). For example, simpler phenyl-substituted compounds that retain a hydrogen-bonding network present in the crystal structure, and further contain an isopropyl group that allows strong hydrophobic interaction with PKC residue Leu 254, bind to PKC, but with lower affinity than ILV. This deficiency may be due to the absence of a hydrophobic interaction between the N-methyl group and the pyrrole ring in ILV with PKC. A naphthyl analog designed to restore this interaction exhibited an 8-fold increase in PKC binding affinity. The essential hydrogen bond network, as well as the hydrophobic interactions of PKC residues Pro 241, Leu 251, Leu 254, provide further opportunity for improving affinity and selectivity of novel PKC activators.

In view of the central role that PKC plays in tumor promotion and signal transduction, PKC is an exciting target for cancer therapy. Oncogenes such as src, ras, and sis, elevate phosphatidylinositol turnover; medical transcription of cellular protooncogenes, including myc, and fos, is mediated by PKC; PKC also regulates the activity of the transcriptional activator protein c-jun, and stimulates the multidrug resistance system. There is increasing evidence that individual PKC isozymes play different, sometimes opposing, roles in biological processes, providing two directions for pharmacological exploitation. One is the design of specific (preferably, isozyme specific) inhibitors of PKC. This approach is complicated by the fact that the catalytic domain is not the domain primarily responsible for the isotype specificity of PKC. Another approach that avoids this problem is to develop isozyme-selective, regulatory site-directed PKC activators. These approaches may provide ways to override the effect of other signal transduction pathways that have opposite biological effects.

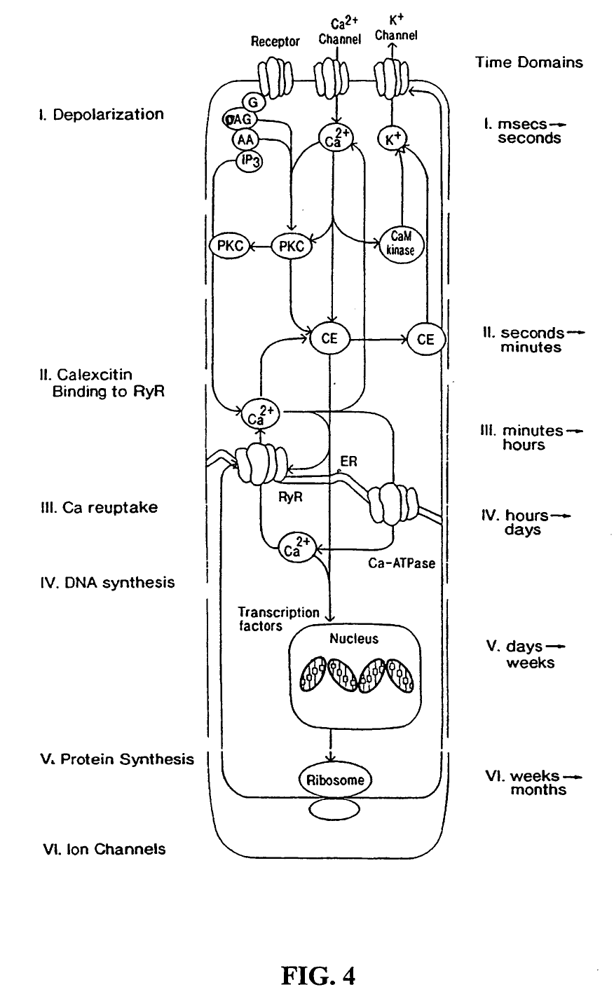

PKC plays an important role in the calcium signaling cascade. This pathway is defective in the cells of patients suffering from Alzheimer's disease (AD) and, thus, correction of defects in the pathway presents a viable treatment for AD. FIG. 4 is a schematic view of various stages in the calcium signaling cascade. The cascade may be envisioned as occurring in the following six stages illustrated in FIG. 2:

Stage I: The neuron depolarizes as a result of a convergence of synaptic input, which activates G-protein coupled receptors (e.g. for acetylcholine, GABA, glutamate). Membrane depolarization also opens Ca²⁺ channels, causing an influx of Ca²⁺. Diacylglycerol (DAG), arachidonic acid (AA), and inositol triphosphate (IP3) are released by phospholipases, and, along with Ca²⁺, activate protein kinase C (PKC), which is thereby translocated to the plasma membrane. Ca²⁺ also activates calmodulin (CaM) kinase. The kinases undergo autophosphorylation, which renders their activity independent of Ca²⁺. PKC and CaM kinase may also inhibit K⁺ and other channels by direct phosphorylation.

Stage II: Elevated Ca²⁺ activates the Ca²⁺-binding protein Cp20, also called calexcitin (CE). Phosphorylation of CE by PKC promotes its translocation to membrane compartments, where it inhibits K⁺ channels, making the membrane more excitable to further depolarizing stimuli. CE also elicits Ca²⁺ release from ryanodine receptors (RyR) on the membrane of the endoplasmic reticulum (ER) and possibly synaptic membranes, resulting in amplification of Ca²⁺ signals.

Stage III: CE, after phosphorylation by PKC, no longer activates the RyR, but activates Ca²⁺-ATPase at the ER membrane, facilitating the removal of excess Ca²⁺.

Stage IV: CE and/or Ca²⁺, probably acting indirectly through transcriptional activators, induce new RNA transcription. CE also increases mRNA turnover.

Stage V: Late genes are transcribed, resulting in increased synthesis of at least 21 different proteins, including RyR. At this stage, retrograde axonal transport is also inhibited by CE; it is believed that this inhibitor may underlie the structural changes in dendritic morphology that are observed after associative learning.

Stage VI: New RyR receptors and ion channels are synthesized and transported to their respective membranes.

A number of the steps of the calcium signaling cascade are defective in AD cells, supporting the notion that calcium homeostasis is involved in the pathophysiology of AD. It is believed that additional defects in the steps of the calcium signaling pathway illustrated in FIG. 2 may exist in AD cells. One important step in the pathway is PKC-mediated phosphorylation of Cp20 (also referred to as CE). Thus, modulation of PKC by activation or deactivation presents a viable treatment for AD.

The 2-pyrrolidone PKC activators of Kozikowski et al. may be employed in treating conditions characterized by the pathological proliferation of mammalian cells (e.g. cancer). In particular, Kozikowski et al. concluded that the 2-pyrrolidones were useful as isozyme selective, non-tumor promoting activators of PKC that cause downregulation and, in the context of cancer treatment, cause initiation of cancer cell death through apoptosis. Thus, selective cancer cell killing may be achieved either through the targeting of those isozymes found to be overexpressed in the cancer cells, or through the synergistic interaction of a cytotoxic drug like 1-D-arabinofuranosylcytosine with an appropriate PKC-based signaling interceptor.

In addition, certain families of benzolactams have been found to have a modulating effect on PKC. For example, PKC can be activated by phorbol esters which significantly increase the relative amount of non-amyloidogenic soluble APP (sAPP) secreted. Activation of PKC by phorbol ester does not appear to result in a direct phosphorylation of the APP molecule, however. Irrespective of the precise site of action, phorbol-induced PKC activation results in an enhanced or favored α-secretase, non-amyloidogenic pathway. Potentially then, PKC activation could be an attractive approach to influence the production of non deleterious and even beneficial sAPP and at the same time reduce the relative amount of Aβ peptides.

Phorbol esters, however, may not be suitable compounds for eventual drug development because of their tumorigenic activity.

Activation of PKC is a multi-step process. Independent of the stimulus, PKC activation involves first, and almost universally, the movement of the enzyme from the cytosol to specific binding domains at cell membranes. This process is termed translocation. For the classic isozymes, an intracellular calcium elevation triggers this event. A subsequent interaction with diacylglycerol (DAG) facilitates penetration into the membrane and complete activation. DAG, although not calcium, is also required to activate the novel isozymes. Translocation does not measure the enzymatic activity per se, i.e. the capacity to phosphorylate a substrate, but it is a prerequisite. Therefore, translocation is a good indicator of the presence of the enzyme in its active form and conformation, thus “biologically ready” to exert its actions. Translocation is commonly and easily measured in the laboratory (Western blots).

The ability of representative compounds of the invention to bind specific isozymes of PKC was determined using an assay (Example 13) in which displacement of [20-³H]phorbol -12,13-dibutyrate ([³H]PDBu) from either recombinant PKC isozymes prepared in a Baculovirus system, or unresolved mixture of isozymes of PKC isolated from whole brain extracts, was used. These procedures are described below. As described in detail below, preferred compounds for neurological effects are those in which affinity for α, β and γ isozymes is the greatest (i.e. those having the lowest Ki) relative to other isozymes as such as the δ- and ε-isozymes shown in Table 1. Preferred compounds for anti-carrier effects are those with high affinities of δ- and ε-isozymes. Results are shown in Table 1. (See the key that follows)

TABLE 1

| | whole brain PKC | α-PKC | β-PKC | γ-PKC | δ-PKC | ε-PKC |
|---|---|---|---|---|---|---|
| Cpd. | (Ki, nM) | (Ki, nM) | (Ki, nM) | (Ki, nM) | (Ki, nM) | (Ki, nM) |
| 1 | >10,000 | 580 ± 214 | 701 ± 217 | 279 ± 50 | 1630 ± 391 | 469 ± 140 |
| | | | | | | 946 ± 274 |
| 2 | >10,000 | 395 ± 71 | 593 ± 178 | 209 ± 38 | 1370 ± 329 | 515 ± 155 |
| 3 | | 674 ± 249 | 1052 ± 284 | 327 ± 65 | 150 ± 64 | 228 ± 43 |
| 4 | | 2251 ± 585 | 8491 ± 3311 | 3649 ± 1095 | 4372 ± 214 | 5101 ± 3111 |
| 5 | | 2980 ± 1043 | 1605 ± 257 | 1229 ± 258 | 623 ± 318 | 1302 ± 377 |
| 6 | | 2253 ± 42% | 4124 ± 31% | 3114 ± 52% | | 1117 ± 38% |
| 7 | | 596 ± 32% | 1285 ± 55% | 2580 ± 30% | | 249 ± 39% |
| 8 | | >10000 | >10000 | >10000 | | 8163 ± 53% |
| 9 | | >10000 | >10000 | >10000 | | 7189 ± 44% |
| 10 | | 2823 ± 29% | 1513 ± 22% | 1320 ± 20% | | 355 ± 34% |
| 11 | | >10000 | >10000 | >10000 | | >10000 |

Compounds of table I have the general structure:

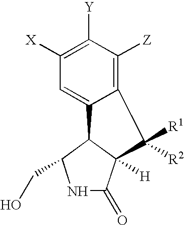

| where: Compo | | | | | |
|---|---|---|---|---|---|
| und | A | B | C | R¹ | R² |
| 1 | H | C≡CC₈H₁₇ | H | CH₃ | i-propyl |
| 2 | H | OC(O)C₁₁H₂₃ | H | CH₃ | i-propyl |
| 3 | H | OC(O)C₁₁H₂₃ | H | i-propyl | CH₃ |
| 4 | C(O)CH₃ | OC(O)C₁₁H₂₃ | H | i-propyl | CH₃ |
| 5 | Br | OC(O)C₁₁H₂₃ | H | i-propyl | CH₃ |
| 6 | Br | OC(O)C₁₁H₂₃ | Br | i-propyl | CH₃ |
| 7 | C≡CC₅H₁₁ | OC(O)C₁₁H₂₃ | H | i-propyl | CH₃ |
| 8 | Br | OC(O)C₁₁H₂₃ | Br | CH₃ | i-propyl |
| 9 | Br | OC(O)C₁₁H₂₃ | H | CH₃ | i-propyl |
| 10 | C≡CC₅H₁₁ | OC(O)C₁₁H₂₃ | H | CH₃ | i-propyl |
| 11 | C(O)CH₃ | OC(O)C₁₁H₂₃ | H | CH₃ | i-propyl |

A compound is specific to a PKC isozyme or group of isozymes if the compound has an affinity for the target isozyme(s) that is higher than the affinity for non-target isozyme or group of isozymes, preferably significantly higher, and if the affinity for the non-target isozyme(s) is as low as possible. As described in more detail below, one group of preferred compounds are those in which affinity for α, β and γ isozymes is the greatest (i.e. those having the lowest Kᵢ) relative to other isozymes such as the δ- and ε-isozymes shown in Table 1. Preferred compounds for specific disease have specificity for particular isozymes. Preferred compounds for neurological treatment have affinities for α, β and γ isozymes that are significantly higher than for other non CNS-specific isozymes. Affinity for γ isozyme is particularly preferred. For prostate cancer, specificity to the δ isoform is preferred. For other disorders, specificity to other isozymes is preferred.

The present inventors have studied benzolactams and flexible 2-pyrrolidones as activators of protein kinase (PKC). Alterations in PKC, as well as alterations in calcium (Ca²⁺) regulation and potassium (K⁺) channels are included among alterations in fibroblasts in Alzheimer's disease (AD) patients. Since PKC is known to regulate ion channels, the present inventors have studied K⁺ channel activity in fibroblasts from AD patients in the presence of (2S, 5S)-8-(1-decynyl)benzolactam (BL), a novel activator of PKC with improved selectivity for the α, β, and δ isozymes. Restoration of normal K⁺ channel function, as measured by TEA-induced [Ca^{2+] elevations, occurs due to activation of PKC by BL. Representative patch-clamp data further substantiate the effect of BL on restoration of} 113pS K⁺ channel activity. Immunoblotting analyses using an β-isozyme-specific PKC antibody confirm that BL-treated fibroblasts of AD patients show increased PKC activation. Thus PKC activator-based restoration of K⁺ channels offers another approach to the investigation of AD pathophysiology, thereby providing a useful model for AD therapeutics.

The use of peripheral tissues from Alzheimer's disease (AD) patients and animal neuronal cells permitted the identification of a number of cellular/molecular alterations that may be the reflection of comparable processes in the AD brain and thus, of pathophysiological relevance (Baker et al., 1988; Scott, 1993; Huang, 1994; Scheuner et al., 1996; Etcheberrigaray & Alkon, 1997; Gasparini et al., 1997). Alterations of potassium channel function have been identified in fibroblasts (Etcheberrigaray et al., 1993) and in blood cells (Bondy et al., 1996) obtained from AD patients. In addition, it was shown that β-amyloid, widely accepted as a major player in AD pathophysiology (Gandy & Greengard, 1994; Selkoe, 1994; Yankner, 1996), was capable of inducing an AD-like K⁺ channel alteration in control fibroblasts (Etcheberrigaray et al., 1994). Similar or comparable effects of α-amyloid on K⁺ channels have been reported in neurons from laboratory animals (Good et al., 1996; also for a review see Fraser et al., 1997). An earlier observation of hippocampal alterations of apamin-sensitive K⁺ channels in AD brains (as measured by apamin binding) provides additional support for the suggestion that K⁺ channels may be pathophysiologically relevant in AD (Ikeda et al., 1991). Furthermore, protein kinase C (PKC) exhibits parallel changes in peripheral and brain tissues of AD patients. The levels and/or activity of this enzyme(s) were introduced in brains and fibroblasts from AD patients (Cole et al., 1988; Van Huynh et al., 1989; Govoni et al., 1993; Wang et al., 1994). Studies using immunoblotting analyses have revealed that of the various PKC isozymes, primarily the α-isozyme was significantly reduced in fibroblasts (Govoni et al., 1996), while both α and β isozymes are reduced in brains of AD patients (Shimohama et al., 1993; Masliah et al., 1990). These brain PKC alterations might be an early event in the disease process (Masliah et al., 1991). It is also interesting to note that PKC activation appears to favor non-amyloidogenic processing of the amyloid precursor protein (APP; Buxbaum et al., 1990; Gillespie et al., 1992; Selkoe, 1994; Gandy & Greengard, 1994; Bergamashi et al., 1995; Desdouits et al., 1996; Efhimiopoulus et al., 1996). Thus, both PKC and K⁺ channel alterations appear to coexist in AD, with peripheral and brain expression in AD.

The link between PKC and K⁺ channel alterations has been investigated. Because PKC is known to regulate ion channels, including K⁺ channels (e.g., see Alkon et al., 1988; Covarrubias et al., 1994; Hu et al., 1996), a defective PKC leads to defective K⁺ channels. To demonstrate this, AD fibroblasts were used in which both K⁺ channels and PKC defects had been independently demonstrated (Etcheberrigaray et al., 1993; Govoni et al., 1993, 1996). Fibroblasts with known dysfunctional K⁺ channels were treated with PKC activators and restoration of channel activity was monitored as presence/absence of TEA-induced calcium elevations. An assay based on tetraethylammonium chloride (TEA)-induced [Ca²⁺] elevation was used because it has been shown to depend on functional 113pS K⁺ channels that are susceptible to TEA blockade (Etcheberrigaray et al., 1993, 1994; Hirashima et al., 1996). Thus, TEA-induced [Ca²⁺] elevations and K⁺ channel activity are primarily observed in fibroblasts from control individuals while being virtually absent in fibroblasts from AD patients (Etcheberrigaray et al., 1993; Hirashima et al., 1996).

It has been demonstrated that the use of a potent novel PKC activator, benzolactam (BL), restored the responsiveness of AD fibroblast cell lines to the TEA challenge. (Bhagavan S, Ibarreta D, Ma D, Kozikowski A P, Etcheberrigaray R., Neurobiol Dis 1998 Sep;5(3):177-87) Immunoblot evidence demonstrates that this restoration is related to a preferential participation of the a isozyme since BL shows improved selectivity for this isozyme that is defective in AD fibroblasts.

The present inventors have also observed that activation of protein kinase C favors the α-secretase processing of the Alzheimer's disease (AD) amyloid precursor protein (APP), resulting in the generation of non-amyloidogenic soluble APP (sAPP). Consequently, the relative secretion of amyloidogenic Aβ₁₋₄₀ and Aβ₁₋₄₂₍₃₎ is reduced. This is particularly relevant since fibroblasts and other cells expressing APP and presenilin AD mutations secrete increased amounts of total Aβ and/or increased ratios of Aβ₁₋₄₂₍₃₎/Aβ₁₋₄₀. Interestingly, PKC defects have been found in AD brain (α, β and γ isozymes) and in fibroblasts (α-isozyme) from AD patients. A novel PKC activator (benzolactam, BL) with improved selectivity for the α, β and γ isozymes to enhance sAPP secretion over basal levels. The sAPP secretion in BL-treated AD cells was also slightly higher compared to control BL-treated fibroblasts, which only showed significant increases of sAPP secretion after treatment with 10 μM BL. Staurosporine (a PKC inhibitor) eliminated the effects of BL in both control and AD fibroblasts. BL causes approximately a 3-fold sAPP secretion in PC12 cells. The use of a novel and possibly non-tumorigenic PKC activator may prove useful to favor non-amyloidogenic APP processing and is, therefore, of potential therapeutic value. Because the concentration of α, β and γ isozymes are higher in the brain in comparison to other tissues, PKC activators that selectively act on α, β and γ isozymes while having a lesser affect on other isozymes are of particular interest. In particular, the PKC α is the most abundant isozyme in the brain and PKC γ is the most specific isozyme for the brain. Such activators of α, β and γ isozymes have the potential to significantly affect PKC in the brain, while having minimal effects on the rest of the body. Thus, for neurological effects, preferred PKC activators are those that have higher selectivity toward α, β and γ isozymes and a lower activity with respect to at least one of the other isozymes. More preferred compounds would have a significantly larger effect on α, β and γ isozymes as compared to other isozymes.

The processing of the amyloid precursor protein (APP) determines the production of fragments that later aggregate forming the amyloid deposits characteristic of Alzheimer's disease (AD), known as senile or AD plaques. Thus, APP processing is an early and key pathophysiological event in AD.

Therefore, although all alternative pathways may take place normally, an imbalance favoring amyloidogenic processing occurs in familial and perhaps sporadic AD. These enhanced amyloidogenic pathways ultimately lead to fibril and plaque formation in the brains of AD patients. Thus, intervention to favor the non-amyloidogenic, β-secretase pathway effectively shifts the balance of APP processing towards a presumably non-pathogenic process that increases the relative amount of sAPP compared with the potentially toxic Aβ peptides.

The invention will now be illustrated by the following non-limiting examples in which, unless otherwise stated: NMR spectra were acquired at proton frequencies of 300 MHz, using CDCl₃ as solvent, unless otherwise noted, ¹H chemical shifts were reported with Me₄Si (δ=0.00 ppm) or CHCl₃ (δ=7.26 ppm) as internal standards, and ¹³C chemical shifts have responded with CHCl₃ (δ=77.0 ppm) or TMS (δ=0.00 ppm) as internal standards. Mass spectra were obtained in electron impact ionization mode at 70 eV. Optical rotations were measured at room temperature.

### EXAMPLES

### Example 1

Referring now to FIG. 2, the pathway for synthesis of (3S, 8R, 9S, 10S)-6-(dec-1′-ynyl)-3-hydroxymethyl-8-isopropyl-8-methyl-3,3a,8,8 tetrahydro-2H-2-aza-cyclopenta[a]inden-1-one (1) is shown. Related compounds of the present invention can also be synthesized by essentially similar methods, and by other methods known to those of skill in the art. Further examples of compounds synthesized by the same general approach represented by Example 1 are given in the remaining examples Modifications necessary to synthesize related compounds will be readily apparent to one of ordinary skill in the art of the present invention. The synthesis of (3S, 8R, 9S, 10S)-6-(dec-1′-ynyl)-3-hydroxymethyl-8-isopropyl-8-methyl-3,3a,8,8a-tetrahydro-2H-2-aza-cyclopenta[a]inden-1-one is described herein to illustrate one embodiment of the present invention, and does not limit the choice of method by which compounds of the present invention are synthesized.

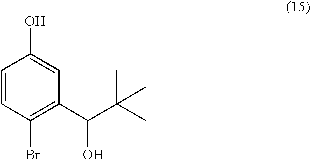

(a) Under nitrogen, to a solution of 2-bromo-5-hydroxybenzaldehyde (14) (8.04 g, 40 m) in 80 mL of anhydrous THF and 20 mL of HMPA at −78° C., is added t-BuMgCl (44 mL, 2.0 M in Hexane, 88 mmol). 3 hours later, this reaction is quenched by 20 mL of MeOH. The resulting mixture is diluted with water (200 mL), and extracted with EtOAc (100 mL×2). The combined organic layer is washed with brine, and dried over MgSO₄. After filtration, the filtrate is concentrated and crystallized from hexane/EtOAc=3/1, giving white solid 6.1 g (59%) (15).

¹H NMR (DMSO-d₆) δ9.55 (s, 1H), 7.28 (d, 1H, J=8.1 Hz), 6.94 (d, 1H, J=3.0 Hz), 6.58 (dd, 1H, J=3.0, 8.1 Hz), 5.31 (d, 1H, J=4.2 Hz), 4.61 (d, 1H, J=4.2 Hz), 0.89 (s, 9H).

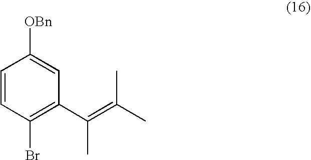

(b) 6.1 g of alcohol (15) (23.5 mmol) is refluxed with BF.₃Et₂O (4.5 mL, 35 mmol) in chlororoform (100 mL) for about 30 min until all starting material is converted. The cooled reaction mixture is washed with water, aq. NaHCO₃, brine, and dried over MgSO₄. After concentration, the residue is benzylated with BnBr (2.86 mL, 24 mmol) and NaH (1.05 g, 25 mmol) in 100 mL of anhydrous DMF. The benzylation is continued for another 2 h, before being quenched carefully with water. The product is extracted with ether (150 mL×2), and the combined organic layer is washed with water, brine, and dried over Na₂SO₄. After concentration, the residue is purified by column chromatography with Hexane and hexane/EtOAc as eluent, to give (16) as a slight yellow oil 6.70 g (86%). ¹H NMR δ7.44−7.38 (m, 6H), 6.72 (m, 2H), 5.02 (s, 2H), 1.87 (s, 3H), 1.80 (s, 3H), 1.43 (s, 3H); ¹³C NMR δ157.98, 146.76, 136.64, 133.07, 129.40, 128.63×2, 128.56, 128.03, 127.57, 127.51×2, 116.41, 114.44, 70.16, 21.69, 19.74, 19.52.

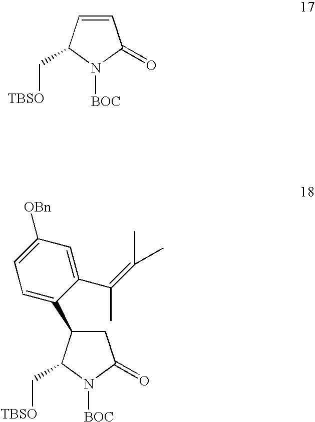

(c) Under N₂, to a freshly prepared Grignard reagent (8.0 mmol of bromide (16) and 8.3 mmol of Mg in 30 mL of THF) at −78° C. is added CuI-Me₂S complex (76 mg of CuI in 1 mL of Me₂S) followed by addition of HMPA (3.0 mL). The resulting mixture is stirred at −78 ° C. for another 20 min before a solution of unsaturated lactam (17) (981 mg, 3 mmol) and TMS (910 μL, 7.2 mmol) in 20 mL of anhydrous THF is added in. This reaction is then continued at −40° C. overnight in a dry ice-acetonitrile bath. After aqueous work-up, the product is extracted with ethyl ether, and purified by flash column chromatography on silica gel with hexane/EtOAc as eluent. The desired product (18) (1.47 g, 85%) is obtained as the mixture of a pair of conformers (ratio 1/1).

α_{D}=−23.6 (c 0.58 in CHCl₃); ¹H NMR (it consists of two rotamors at 1/1 ratio) 6 7.44−7.32 (m, 5H), 7.09 & 7.07 (d, 1H, J=8.1 Hz), 6.85 (br d, 1H, J=8.1 Hz), 6.62 (t, 1H, J=2.4 Hz), 5.02 (s, 2H), 4.03 (br s, 1H), 4.00 (br t, 0.5H, J=3 Hz), 3.92 (br s, 0.5H), 3.63 (AB q, 0.5H, J=9.3 Hz), 3.50 (AB q, 0.5H, J=10.8 Hz), 3.47 (d, 0.5H, J=12.3 Hz), 3.38 (d, 0.5 H, J=9.9 Hz), 3.12 (AB qd, 0.5H, J=9.9, 18.0 Hz), 3.05 (AB qd, 0.5H, J=9.9, 18.0 Hz), 2.42 (AB qd, 0.5H, J=1.8, 18.0 Hz), 2.33 (AB qd, 0.5H, J=2.7, 18.0 Hz), 1.86 (br s, 3H), 1.80 (s, 3H), 1.53 (s, 4.5H), 1.52 (s, 4.5H), 1.41 (s, 1.5H), 1.38 (s, 1.5H), 0.89 (s, 9H), 0.05 (s, 3H), 0.04 (s, 3H);

¹³C NMR δ174.76, 157.49, 149.99, 145.26, 145.16, 136.92, 134.72, 134.61, 128.53, 127.93, 127.54, 126.04, 115.11, 114.74, 113.70, 113.53, 82.91, 82.83, 69.97, 66.96, 64.25, 63.9640.01, 40.79, 34.04, 33.55, 28.08, 25.83, 22.24, 22.02, 21.24, 20.75, 19.68, 18.23, −5.54, −5.68.

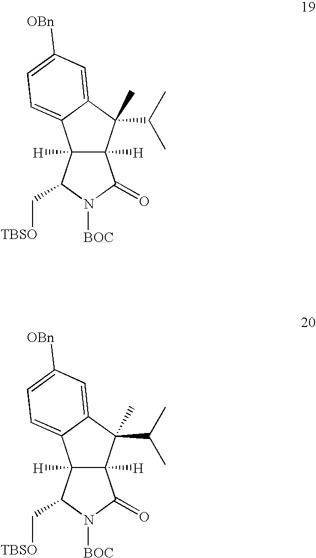

d) Under N₂, the product from last step (18) (319 mg, 0.552 mmol) is treated with LiHMDSA (0.662 mmol) in THF at −78° C. for 1 h before addition of PhSeCl (108 mg, 0.552 mmol). After another hour, the product is extracted with ether and purified by column chromatography, giving 336 mg of phenyl selenyl ether (83%). Under N₂, a solution of Bu₃SnH (240 ML, 0.91 mmol) and AIBN (20 mg) in toluene (10 mL) is added dropwise over 2 h via a syringe pump to a refluxing solution of phenyl selenyl ether (336 mg, 0.458 mmol) in 20 mL of toluene. After addition, the reaction mixture is concentrated, and the products are separated by careful column chromatography, giving cyclized product 1 (19) (132 mg, 41%), the epimeric cyclized product 2 (20) (82 mg, 26%), recovered starting material (18) (40 mg, (12%). The stereochemistry of lactams (19) and (20) were assigned by ¹H—¹H COSY and ¹H—¹H NOESY as well as by X-ray diffraction of lactam (20). α_{D}=−6.1 (c 0.53 in CHCl₃). ¹H NMR δ7.28 (dd, 1H, J=1.2, 7.8 Hz), 7.14 (d, 1H, J=7.8 Hz), 7.13 (br s, 1H), 4.11 (br s, 1H), 4.01 (dd, 1H, J=3.3, 10.2 Hz), 3.85 (dd, 1H, J=1.8, 10.2 Hz), 3.67 (d, 1H, 8.7 Hz), 3.15 (d, 1H, J=8.4 Hz), 2.39 (t, 2H, J=6.9 Hz), 1.74 (m, 1H), 1.60 (m, 2H), 1.47 (s, 9H) 1.41 (s, 3H), 1.29 (br s, 12 H), 0.96 (d, 3H, J=6.6 Hz), 0.91 (br s, 15 H), 0.64 (d, 3H, J=6.6 Hz, 0.09 (s, 3H), 0.07 (s, 3H);

¹³C NMR δ173.82, 150.57, 150.25, 142. 42, 130.88, 126.58, 123.50, 123.44, 90.22, 82.55, 80.59, 64.61, 63.88, 54.78, 52.34, 44.55, 39.37, 31.84, 29.19, 29.12, 28.95, 28.77, 28.07×3, 25,82×3, 22.66, 21.99, 19.41, 18.12, 18.08, 17.82, 14.11, −5.52, −5.54.

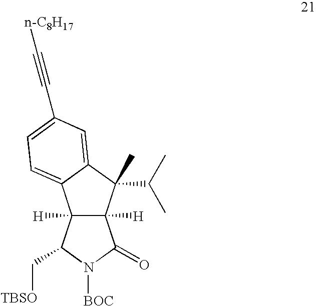

f) Debenzylation of cyclized product 1 (19) (41 mg) is achieved via hydrogenation over 10% Pd/C in MeOH, giving 27.1 mg of phenol. 8.0 mg of starting material is recovered. The yield is 97% based on converted starting material. This phenol (27.1 mg, 55.5 μmol) is triflated with trifluoromethanesulfonic anhydride and 2,6-lutidine (50 μL) in 1.4 mL of CH₂Cl₂ at −78° C. The product is purified by flash column chromatography with hexane/EtOAc (v/v 10/1) as eluent, giving 32.3 mg of triflate (96%). A mixture of triflate (32.3 mg, 52 μmol), CuI (4 mg), PdCl₂(PPh₃)₂ (15 mg, 10.6 μmol), 1-decyne (100 μL), and Et₃N (1 mL) is stirred at 70° C. in 1 mL of DMF under Argon for 24 h. The reaction mixture is extracted with ethyl ether, and washed with water, brine, and dried over Na₂SO₄. After careful column chromatography, the desired product (21) is obtained as an oil (24.5 mg, 77%).

¹H NMR δ7.28 (dd, 1H, J=1.2, 7.8 Hz), 7.14 (d, 1H, J 7.8 Hz), 7.13 (br s, 1H), 4.11 (br s, 1H), 4.01 (dd, 1H, J=3.3, 10.2 Hz), 3.85 (dd, 1H, J=1.8, 10.2 Hz), 3.67 (d, 1H, 8.7 Hz), 3.15 (d, 1H, J=8.4 Hz), 2.39 (t, 2H, J=6.9 Hz), 1.74 (m, 1H), 1.60 (m, 2H), 1.47 (s, 9H) 1.41 (s, 3H), 1.29 (br s, 12 H), 0.96 (d, 3H, J=6.6 Hz), 0.91 (br s, 15 H), 0.64 (d, 3H, J=6.6 Hz, 0.09 (s, 3H), 0.07 (s, 3H);

¹³C NMR δ173.82, 150.57, 150.25, 142. 42, 130.88, 126.58, 123.50, 123.44, 90.22, 82.55, 80.59, 64.61, 63.88, 54.78, 52.34, 44.55, 39.37, 31.84, 29.19, 29.12, 28.95, 28.77, 28.07×3, 25,82×3, 22.66, 21.99, 19.41, 18.12, 18.08, 17.82, 14.11, −5.52, −5.54.

(g) Desilylation of the product from last step (21) (24.5 mg) is achieved with TBAF (0.2 mL, 1.0 M in THF) in 0.5 ml of THF, giving primary alcohol 18.8 mg (94%). The Boc group is removed with 0.5 mL of TFA in 0.5 mL of dichloromethane, giving colorless final product (1) (80%).

α_{D}=−83.1 (c 0.50 in CHCl₃);

¹H NMR δ7.25 (d, 1H, J=7.8 Hz), 7.12 (br s, 1H), 7.09 (d, 1H, J=7.8 Hz), 6.69 (br s, 1H), 3.86 (dd, 1H, J=3.0, 11.1 Hz), 3.70−3.58 (m, 3H), 3.05 (d, 1H, J=9.3 Hz), 2.39 (t, 2H, J=6.9 Hz), 1.85 (m, 1H), 1.60 (m, 2H), 1.45 (m, 2H) 1.37 (s, 3H), 1.29 (br s, 8 H), 0.99 (d, 3H, J=6.6 Hz), 0.89 (t, 3H, J=6.9 Hz), 0.60 (d, 3H, J=6.6 Hz);

¹³C NMR δ178.99, 150.50, 142.42, 130.87, 126.62, 123.46, 123.35, 90.17, 80.62, 66.25, 62.24, 54.25, 50.05, 47.32, 38.67, 31.84, 29.18, 29.12, 28.96, 28.77, 23.31, 22.66, 19.41, 18.10, 17.87, 14.11; EI-MS: 395 (M⁺, 2%), 352 (94%), 291 (12%), 267 (14%), 181 (38%), 155 (76%), 43 (100%). The synthesis of Examples 2-11 is described in FIG. 3 and Example 12.

### Example 2

(3S, 8R, 9S, 10S)-6-dodecanoyloxyl-3-hydroxymethyl-8-isopropyl-8-methyl-3,3a,8,8a-tetrahydro-2H -2-aza-cyclopenta[a]inden-1-one. [α]_{D}=−80.8 (c 0.33 in CHCl₃):

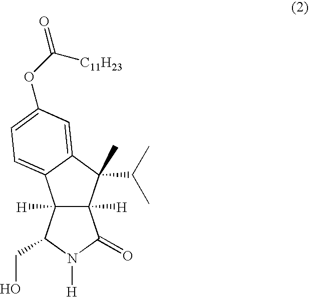

¹H NMR δ7.16 (d, 1H, J=7.8 Hz), 6.94 (dd, 1H, J=2.1, 8.1 Hz), 6.79 (d, 1H, J=1.8 Hz), 6.24 (s, 1H), 3.87 (dd, 1H, J=3.0, 10.8 Hz), 3.71−3.60 (m, 3H), 3.09 (d, 1H, J=9.0 Hz), 2.90 (br s, 1H), 2.54 (t, 2H, J=7.8 Hz), 1.39 (s, 3H), 1.35−1.27 (br s, 18 H), 1.02 (d, 3H, J=6.6 Hz), 0.89 (t, 3H, J=8.1 Hz), 0.64 (d, 3H, J=6.6 Hz);

¹³C NMR δ172.42, 152.091, 150.70, 140.01, 124.13, 120.81, 116.68, 77.20, 66.41, 61.92, 54.37, 47.00, 38.70, 34.41, 31.90, 29.60, 29.45, 29.32, 29.26, 29.11, 24.90, 23.15, 22.68, 18.13, 17.85, 14.12.

### Example 3

(3S, 8S, 9S, 10S)-6-dodecanoyloxyl-3-hydroxymethyl-8-isopropyl-8-methyl-3,3a,8,8a-tetrahydro-2H -2-aza-cyclopenta[a]inden-1-one

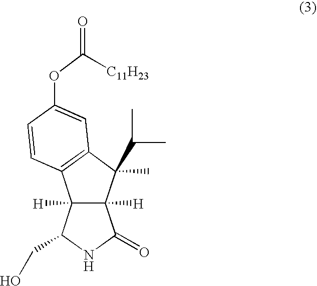

¹H NMR δ7.19(d, 1H, J=8.1 Hz), 6.94(dd, 1H, J=1.8, 8.1 Hz), 6.92(brs, 1H), 6.21(br s, 1H), 3.90(dd, 1H, J=3.0, 11.4 Hz), 3.71(m, 3H), 3.05(d, 1H, J=9.9 Hz), 2.55(t, 2H, J=7.5 Hz), 2.46-2.60(m, 1H), 1.60-1.80(m, 5H), 1.38(s, 3H), 1.27(br s, 14H), 1.07(d, 3H, J=7.2 Hz), 0.89(d, 3H, J=6.3 Hz), 0.88(t, 3H, J=6.9 Hz).

¹³C NMR δ178.55, 172.67, 15150, 150.57, 139.96, 124.64, 120.89, 117.72, 66.56, 61.32, 57.21, 55.28, 46.41, 34.65, 32.89, 32.13, 31.01, 29.83, 29.69, 29.56, 29.49, 29.35, 25.13, 22.92, 19.72, 19.29, 14.35.

### Example 4

(3S, 8S, 9S, 10S)-5-acetyl-6-dodecanoyloxyl-3-hydroxymethyl-8-isopropyl-8-methyl-3,3a,8,8a-tetrahydro-2H -2-aza-cyclopenta[a]inden-1-one

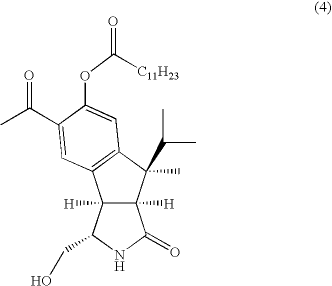

¹H NMR δ7.65(s, 1H), 6.95(s, 1H), 6.22(br s, 1H), 3.91(dd, 1H, J=6.0, 12.3 Hz), 3.76(m, 3H), 3.07(d, 1H, J=9.6 Hz), 2.73(br s, 1H), 2.62(t, 2H, J=7.5 Hz), 2.54(s, 3H), 2.50-2.67(m, 1H), 1.60-1.82(m, 4H), 1.38(s, 3H), 1.27(br s, 14H), 1.09(d, 3H, J=7.2 Hz), 0.93(d, 3H, J=6.9 Hz), 0.89(t, 3H, J=6.9 Hz).

¹³C NMR δ197.56, 178.03, 172.52, 156.27, 149.33, 140.61, 130.47, 125.68, 119.79, 66.43, 61.10, 56.93, 55.64, 46.34, 34.67, 32.13, 31.18, 30.78, 29.92, 29.83, 29.69, 29.56, 29.50, 29.38, 24.75, 22.91, 19.67, 19.28, 14.35.

### Example 5

(3S, 8S, 9S, 10S)-5-bromo-6-dodecanoyloxyl-3-hydroxymethyl-8-isopropyl-8-methyl-3,3a,8,8a-tetrahydro-2H -2-aza-cyclopenta[a]inden-1-one

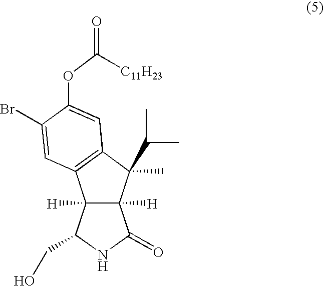

¹H NMR δ7.43(s, 1H), 6.95(s, 1H), 6.60(br s, 1H), 3.85(m, 1H), 3.64-3.78(m, 3H), 3.32(br s, 1H), 3.04(d, 1H, J=9.3 Hz), 2.61(t, 2H, J=7.5 Hz), 2.50(hept, 1H, J=6.9 Hz), 1.79(m, 2H), 1.35(s, 3H), 1.27(br s, 16H), 1.05(d, 3H, J=6.9 Hz), 0.88(m, 6H).

¹³C NMR δ178.52, 171.73, 150.89, 147.84, 142.12, 128.57, 119.70, 114.87, 66.15, 61.35, 57.31, 55.17, 46.26, 34.38, 32.88, 32.12, 30.89, 29.82, 29.68, 29.55, 29.46, 29.35, 25.02, 22.91, 19.71, 19.26, 14.35.

### Example 6

(3S, 8S, 9S, 10S)-5,7-dibromo-6-dodecanoyloxyl-3-hydroxymethyl-8-isopropyl-8-methyl-3,3a,8,8a-tetrahydro-2H-2-aza-cyclopenta[a]inden-1-one

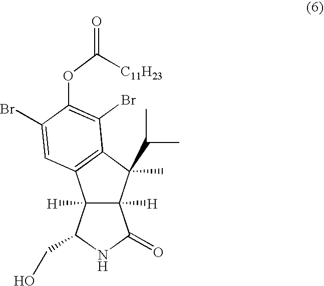

¹H NMR δ7.42(s, 1H), 6.65(br s, 1H), 3.83(m, 1H), 3.60-3.78(m, 3H), 3.47(br s, 1H), 3.06(d, 1H, J=9.0 Hz), 3.00(hept, 1H, J=6.9 Hz), 2.66(t, 2H, J=7.5 Hz), 1.81(pent, 2H), 1.50(s, 3H), 1.20-1.50(m, 19H), 0.88(m, 6H).

¹³C NMR δ178.20, 170.57, 149.33, 146.24, 144.03, 127.52, 116.20, 115.51, 65.92, 60.63, 60.24, 56.01, 46.44, 34.17, 32.92, 32.12, 29.81, 29.67, 29.55, 29.45, 29.36, 28.98, 28.68, 24.90, 22.90, 18.64, 17.48, 14.34.

### Example 7

(3S, 8S, 9S, 10S)-6-dodecanoyloxyl-5-(hept-1′-ynyl)-3-hydroxymethyl-8-isopropyl-8-methyl-3,3a,8,8a-tetrahydro-2H -2-aza-cyclopenta[a]inden-1-one

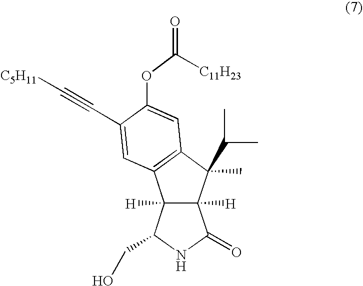

¹H NMR δ7.27(s, 1H), 6.89(s, 1H), 6.29(br s, 1H), 3.86(m, 1H), 3.62-3.76(m, 3H), 3.02(d,1H, J=9.9 Hz), 2.86(br s, 1H), 2.57(t, 2H, J=7.5 Hz), 2.50(hept, 1H, J=6.9 Hz), 2.38(t, 2H, J=7.5 Hz), 1.65-1.84(m, 4H), 1.58(pent, 2H, J=6.9 Hz), 1.20-1.48(m, 21H), 1.06(d, 3H, J=6.9 Hz), 0.82-0.96(m, 9H).

¹³C NMR δ178.47, 172.03, 151.40, 151.08, 140.23, 128.31, 118.30, 117.17, 95.38, 75.79, 66.39, 61.32, 57.20, 55.38, 46.28, 34.53, 33.04, 32.13, 31.33, 30.89, 29.86, 29.72, 29.57, 29.46, 28.68, 25.18, 22.91, 22.47, 19.77, 19.70, 19.27, 14.35, 14.21.

### Example 8

(3S, 8R, 9S, 10S)-5-bromo-6-dodecanoyloxyl-3-hydroxymethyl-8-isopropyl-8-methyl-3,3a,8,8a-tetrahydro-2H-2-aza-cyclopental[a]inden-1-one

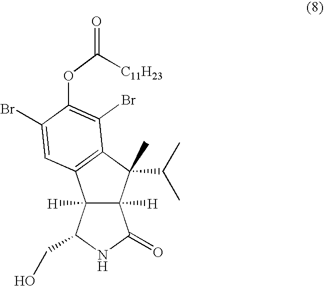

¹H NMR δ7.41(s, 1H), 6.83(s, 1H), 6.59(br s, 1H), 3.84(dd, 1H, J=2.7, 11.8 Hz), 3.56-3.76(m, 3H), 3.47(br s, 1H), 3.09(d, 1H, J=9.0 Hz), 2.60(t, 2H, J=7.5 Hz), 1.78(m, 4H), 1.37(s, 3H), 1.27(br s, 15H), 0.99(d, 3H, J=6.6 Hz), 0.88(t, 3H, J=6.9 Hz), 0.65(d, 3H, J=6.6 Hz).

¹³C NMR 178.63, 171.69, 151.72, 148.18, 142.30, 128.28, 118.99, 114.91, 66.24, 62.15, 54.50, 50.66, 47.09, 38.90, 34.38, 32.12, 29.82, 29.68, 29.56, 29.47, 29.35, 25.03, 23.17, 22.91, 18.31, 18.02, 14.35.

### Example 9

(3S, 8R, 9S, 10S)-5,7-dibromo-6-dodecanoyloxyl-3-hydroxymethyl-8-isopropyl-8-methyl-3,3a,8,8a-tetrahydro-2H-2-aza-cyclopental[a]inden-1-one:

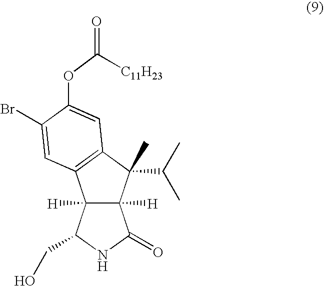

### Example 10

(3S, 8R, 9S, 10S)-6-dodecanoyloxyl-5-(hept-1′-ynyl)-3-hydroxymethyl-8-isopropyl-8-methyl-3,3a,8,8a-tetrahydro-2H -2-aza-cyclopenta[a]inden-1-one

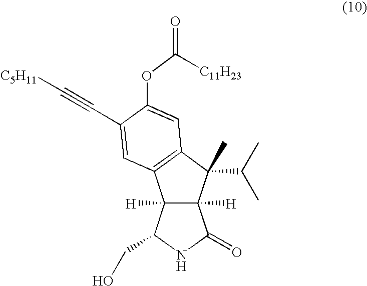

¹H NMR δ7.27(s, 1H), 6.77(s, 1H), 6.27(br s, 1H), 3.85(m, 1H), 3.56-3.73(m, 3H), 3.08(d, 1H, J=9.0 Hz), 3.00(br s, 1H), 2.58(t, 2H, J=7.8 Hz), 2.39(t, 2H, J=7.2 Hz), 1.72-1.86(m, 3H), 1.58(pent, 2H, J=7.2 Hz), 1.20-1.50(m, 23H), 1.01(d, 3H, J=6.6 Hz), 0.92(t, 3H, J=7.2 Hz), 0.89(t, 3H, J=6.9 Hz), 0.64(d, 3H, J=6.9 Hz).

¹³C NMR δ178.53, 172.00, 151.95, 151.79, 140.48, 128.09, 117.52, 117.30, 95.24, 75.85, 66.45, 62.11, 54.71, 50.46, 47.09, 38.88, 34.52, 32.13, 31.32, 31.17, 29.86, 29.84, 29.71, 29.57, 29.46, 28.69, 25.19,23.27, 22.91, 22.47, 19.77, 18.33, 18.05, 14.34, 14.20.

### Example 11

(3S, 8R, 9S, 10S)-5-acetyl-6-dodecanoyloxyl-3-hydroxymethyl-8-isopropyl-8-methyl-3,3a,8,8a-tetrahydro-2H -2-aza-cyclopenta[a]inden-1-one

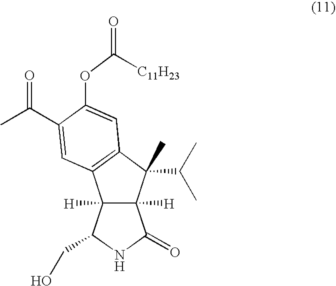

¹H NMR δ7.64(s, 1H), 6.82(s, 1H), 6.43(br s, 1H), 3.88(m, 1H), 3.68-3.78(m, 2H), 3.60-3.68(m, 1H), 3.22(br s, 1H), 3.13(d, 1H, J=9.9 Hz), 2.61(t, 2H, J=7.5 Hz), 2.54(s, 3H), 1.68-1.90(m, 3H), 1.40(s, 3H), 1.27(br s, 16H), 1.02(d, 3H, J=6.9 Hz), 0.89(t, 3H, j=6.6 Hz), 0.66(d, 3H, J=6.6 Hz).

¹³CNM R δ197.59, 178.38, 172.51, 157.05, 149.70, 140.87, 130.67, 125.47, 119.10, 66.41, 62.07, 54.93, 50.52, 47.17, 38.87, 34.66, 32.12, 29.90, 29.82, 29.68, 29.55, 29.49, 29.36, 24.74, 23.01, 22.90, 18.32, 17.99, 14.34.

### Example 12

The introduction of multi-substituents at the positions next to the benzyloxy group of intermediate (19) and (20) were complemented through two strategies, photochemical Fries rearrangement and bromination. When acetate (23) was subject to UV irradiation under 254 nM in the presence of catalytic amount of K₂CO₃ in hexane, the acetyl group migrated to the less steric hindrance ortho-position. This structure was confirmed by observation of two singlet peaks for two aromatic protons of compound 11 in ¹H NMR. Following the similar procedures shown above, compound (25) was obtained as a derivative of lactam (20) with a side arm next to the ester appendage. In the same manner was the diastereoisomer (4) synthesized.

Mono-bromination of phenol (27) derived from intermediate (19) with 1 eq. of NBS in anhydrous acetonitrile provided bromide (28), favoring the less steric hindrance ortho-position. Subsequent esterification and usual deprotection led to the monobromo-derivative (9). The bromide may be converted into a lipophilic decynyl chain via palladium-mediated coupling reaction. Double decynyl chains were also achieved using the same coupling reaction through a triflate intermediate derived from phenol (27). Final deprotection furnished compound (30) and (31) respectively. In the same manner, the diastereoisomers of pyrrolidones (5), (30), and (31) were also prepared from intermediate (20).

Reagents and conditions in FIG. 3: (a) NBS, CH₃CN, rt, 1 h, 95%. (b) lauroyl chloride, pyridine, DMAP (cat.), CH₂Cl₂, rt, overnight, 90%. (c) 48% HF, CH3CN, rt, overnight, 95%. (d) 1-heptyne, PdCl₂(PPh₃)₂ (5% mol), CuI (10% mol), n-Bu₄NI, Et₃N, DMF, 80° C., 2 days, 80%. (e) Tf₂O, 2,6-lutidine, CH₂Cl₂, −78° C. to −40° C., 90%;%. (g) 1-decyne, PdCl₂(PPh₃)₂ (5% mol), CuI (10% mol), n-Bu₄NI, Et₃N, DMF, 80° C., 2 days, 80%.

Dibromonation at two ortho positions of phenol (27) took place quantitatively when 2 equivalent of NBS was used under the same bromination condition. With the notion of the electronic property of the phenyl ring that might somehow influence the PKC binding affinity, dibromo-pyrrolidone (6) and its diastereoisomer (8) were prepared.

### Example 13

Analysis of [20-³H]phorbol 12,13-dibutyrate binding to PKC. Specific binding of [³H]PDBu to PKC was carried out as described (Kazanietz, et al., Mol. Pharmacol. 44:298-307 (1993)). [³H]PDBu (20.0 Ci/mmol) was obtained from New England Nuclear. Phosphatidylserine and bovine δ-globulin were obtained from Sigma Chemical Co. Binding was determined in an incubation volume of 250 μl, containing [³H]PDBu, PKC, 0.05 M Tris-Cl, pH 7.4, 100 μg/ml phosphatidylserine, various concentrations of the ligand being assayed for competition of [³H]PDBu binding, and bovine x-globulin at 1 mg/ml. In the case of the classical PKCs (α, β, δ) CaCl₂ was included at a concentration of 0.1 mM. Liposomes of phosphatidylserine were prepared as follows. The phosphatidylserine solutions were dried down from CHCl₃ under a stream of N₂. After addition of 0.05 M Tris-Cl, pH 7.4, liposomes were prepared by sonication for 30 sec with the microtip probe of Sonifier Cell Disrupter (Heat Systems-Ultrasonics). [³H]PDBu was used at a concentration twice the K_{d} previously determined for the specific receptor. Ligands were assayed over a concentration range of 3 orders of magnitude. Ligands were diluted in 0.05 M Tris-Cl, pH 7.4 containing 1 mg/ml bovine g-globulin. If the compounds were not soluble at the highest concentration used, they were dissolved in organic solvent (CHCl₃ or ethanol) and mixed with the phosphatidylserine dissolved in CHCl₃. The solvent was removed under a stream of N₂.

Mixed liposomes were then prepared, as described for the preparation of the liposomes of phosphatidylserine alone, by the addition of 0.05 M Tris-Cl, pH 7.4, and sonication for 30 sec with the microtip probe of a Sonifier Cell Disrupter (Heat Systems-Ultrasonics). Incubation was for 5 min at 37° C. The samples were then be cooled to 4° C. for 8 min and 200 μl of 35% polyethylene glycol in 0.05 M Tris-Cl, pH 7.4 (w:w) added. After vigorous mixing, the samples were incubated for 15 min to allow precipitation of the receptor. The precipitate was recovered by centrifugation at 12,000 rpm in a Beckman microcentrifuge at 4° C. A 100 μl aliquot of the supernatant was removed and its radioactivity determined in a Wallac liquid scintillation counter, Model 1409. This value provided a measure of the concentration of unbound [³H]PDBu. The remainder of the supernatant was removed by aspiration and blotting with absorbent tissue. The tip of the centrifuge tube was cut off and the radioactivity in the pellet measured to determine total bound [³H]PDBu. Non-specific binding of [³H]PDBu was measured in the presence of 30 μM non-radioactive PDBu and used to determine an apparent partition coefficient for [³H]PDBu under these assay conditions.

Specific binding represents the difference between total binding and the non-specific binding, where the latter value was calculated from the partition coefficient and the measured free [³H]PDBu concentration in each tube. In each experiment, triplicate determinations were performed at each concentration of competing ligand. A half-maximal inhibitory concentration of inhibitor and a Kᵢ for the inhibitor was determined by computer fit of the data points to the equation B(I)/B(O)=(L+K_{d})/(K_{d}+(K_{d}/Kᵢ)(I)+L), where B(I) represents the amount of [³H]PDBu specifically bound in the presence of inhibitor, B(O) represents the amount specifically bound in the absence of inhibitor, L is the concentration of [³H]PDBu, K_{d} is the measured dissociation constant for [³H]PDBu, Kᵢ is the dissociation constant for the inhibitor, and I is the concentration of inhibitor. For each inhibitor, three experiments were performed to confirm the reproducibility of the measured Kᵢ.

Expression of PKC isozymes in baculovirus. The PKC isozymes are expressed in the baculovirus system and partially purified as has been previously described.(Kazanietz, et al., Mol. Pharmacol. 44:298-307 (1993); Areces, et al., J. Biol. Chem. 269:19553-19558 (1994); Caloca, et al., J. Biol. Chem. 272:26488-26496 (1997)). Briefly, 1 liter of approximately 2×106 Sf9 cells/ml in spinner flasks was infected with the recombinant viruses at a multiplicity of infection of 10. After 60-72 hr, the cells were centrifuged (1000 rpm, 10 min) and washed twice with phosphate buffered saline, and the pelleted cells kept at −70° C. until used.

For purification of the individual PKC isozymes, the cell pellet was resuspended in 50 mnl of a homogenization buffer of the following composition; 20 mM Tris-Cl, pH 8.0, 5 mM EDTA, 5 mM EGTA, 0.3% (v/v) 2-mercaptoethanol, 10 mM benzamidine, 50 μg/ml phenylmethylsulfonyl fluoride, and 250 μg/ml leupeptin. The cells were disrupted in a Potter-Elvehjem homogenizer at 4° C., and the homogenate is centrifuged at 100,000×g for 60 min. The supernatant was adjusted to pH 8.0 and loaded in a TSK-GEL DEAE-5PW column (15 cm×2 cm; Tosohaas, Philadelphia, Pa.) that was equilibrated with 20 mM Tris-Cl, pH 7.5, 2 mM EDTA, 0.3% 2-mercaptoethanol, 10 mM benzamidine. The colunm was eluted with a 375 ml linear gradient of NaCl (0-400 mM) in the equilibration buffer at a flow rate of 2.5 ml/min. Fractions were dialyzed against 20 mM Tris-Cl, pH 7.5, 1 mM dithiothreitol, 1 mM EDTA, 50% glycerol, before storage at −70° C.

Because of their ability to activate PKC, compounds of the invention are useful for treating diseases or conditions wherein PKC activity is implicated and wherein activation of PKC is desirable. For example, compounds of the invention may be useful for treating a disease or condition characterized by the pathological proliferation of mammalian cells, such as for example, human cancers, such as prostate cancer, solid tumors and leukemias. Compounds of the invention may also be useful for treating autoimmune diseases, neurological disorders such as Alzheimer's disease, and inflammation.

Accordingly, the invention includes a method comprising modulating PKC in a mammal by administering to said mammal a pharmaceutically effective dose of a compound of formula I; or a pharmaceutically acceptable salt thereof. The invention also provides a method comprising treating a condition characterized by the pathological proliferation of mammalian cells by administering to a mammal afflicted with such a condition, an effective amount of a compound of formula I; or a pharmaceutically acceptable salt thereof.

Examples 14 and 15 utilize methods which have been previously described with respect to benzolactams. Ibarreta D, Duchen M, Ma D, Qiao L, Kozikowski A P, Etcheberrigaray R., “Benzolactam (BL) enhances sAPP secretion in fibroblasts and in PC₁₂ cells.” Neuroreport 1999 Apr 6;10(5):1035-40; Bhagavan S, Ibarreta D, Ma D, Kozikowski A P, Etcheberrigaray R., “Restoration of TEA-induced calcium responses in fibroblasts from Alzheimer's disease patients by a PKC activator.” Neurobiol Dis 1998 Sep;5(3):177-87. These results have now been found extended remarkably to certain rigid pyrrolidones.

### Example 14

Rigid Pyrrolidone Derivatives as α-Secretase Promotors

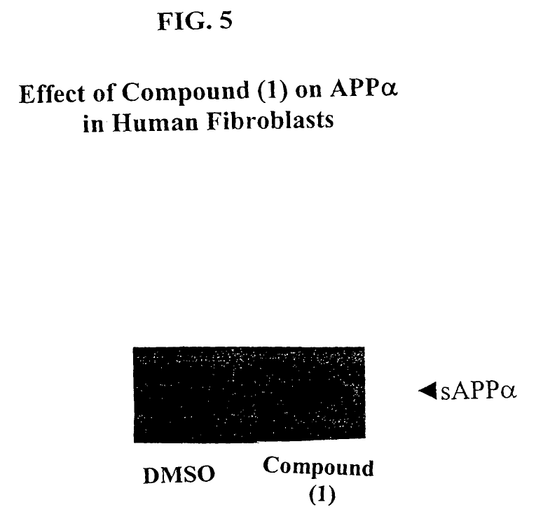

Research on cellular and molecular aspects of Alzheimer's disease (AD) over the last two decades suggested that the metabolic fate of the amyloid precursor protein (APP) is one of the key factors in the pathogenesis of AD. Most of the experimental evidence suggested that the relative ratio of amyloidogenic vs. non-amyloidogenic products could be altered by regulating the activity of α and β-secretases in the processing of APP. A growing list of evidence implicated that protein kinase C (PKC) activation can increase the rate of the non-amyloidogenic, i.e. α-secretase processing in vitro. Preliminary results FIG. 5 showed that compound (1) is able to cause the translocation of PKC catalytic subunit from the cytosolic to the membrane fractions (Activation). This compound also increased the secretion of sAPP in human fibroblasts (AD patients).

Translocation of PKC Catalytic Subunit to the Membrane Fraction:

Human fibroblasts were grown to confluence in 100 mm tissue culture dishes. On the day of the experiment, cells were rinsed twice with serum-free Dulbecco's modified Eagle's medium (DMEM) and incubated in the same medium for 2 hours. Cells were treated with the drugs above, or DMSO vehicle as control, for 15 min. The cells were then rinsed twice with ice-cold phosphate buffered saline (PBS), scraped in PBS, and collected by centrifugation. The pellets were suspended in homogenization buffer containing: 20 mM Tris-HCl, pH 7.5, 2 mM EDTA, 2 mM EGTA, 5 mM DTT, 0.32 M sucrose, 2 mM PMSF, 25 g/ml aprotinin, and 20 g/ml leupeptin. Homogenates were immersed in liquid nitrogen thawed, and centrifuged at 12,000 g for 20 min, and supernatants were used as the cytosolic fraction (C). The pellets were homogenized in the same buffer containing 1.0% Triton X-100, incubated on ice for 45 min, and centrifuged at 12,000 g for 30 min and the supernatants were used as the membrane fractions (M). After protein determination, equal amounts of proteins (approx. 20 g) were mixed with electrophoresis buffer, boiled for 5 min, and separated on 8% SDS-PAGE, and electrophoretically transferred to PVDF membranes. The membranes were processed by Western blotting using a specific antibody for PKC isozyme.

Human fibroblasts were grown to confluence as above for PKC translocation. On the day of the experiment, cells were rinsed twice with serum-free Dulbecco's modified Eagle's medium (DMEM) and incubated in the same medium for 2 hours. Cells were treated with the drugs above, or DMSO vehicle as a control, for 2 hours. The medium was collected and spun for 5 min in a clinical centrifuge to remove cell debris. Proteins in the cell medium were precipitated using 10% trichloroacetic acid (TCA). Precipitated proteins were dissolved in electrophoresis buffer, separated on 8% SDS-PAGE as described above. Secreted APP was detected by Western blotting using a specific monoclonal antibody, 6E10. FIG. 5 shows a western blot demonstrating the effect of compound (1) on sAPP α. Compound 1 increases sAPPα concentration relative to a DMSO control.

### Example 15

Effect of Rigid Pyrrolidones on the Secretion of Soluble APPα from Human Fibroblasts

Human fibroblasts from Alzheimer's patients (AD, cell line AG06848) or age-matched controls (AC, cell line AG07141) were grown to near confluence in DMEM containing 10% fetal bovine serum. On the day of the experiments, cells were rinsed twice with DMEM with no serum and incubated with the same method for 2 hours at 37° C., 5% CO₂. Cells were incubated for 2 additional hours with different concentrations of the drugs or equal volume of DMSO vehicle as control. The medium was collected and centrifuged at 1000 rpm to remove cell debris. Proteins were precipitated by addition of equal volume of 20% ice-cold tricholoroacetic acid (TCA) and incubation on ice for 60 minutes. The protein pellets were washed with ice-cold acetone and centrifuged for 30 minutes at 20,000 g for 30 minutes and dried overnight. Pellets were dissolved in electrophoresis buffer and proteins corresponding to the same concentration of protein were separated by SDS-PAGE and transferred to PVDF membranes according to the manufacturer's instructions. After electrophoresis, the membranes were blocked with Blotto and incubated overnight with a monoclonal antibody 6E10 (Synetek) that recognizes the APPα. After incubation with the secondary antibody, alkaline phosphatase anti-mouse IgG (Jackson's Laboratories), the membranes were developed using an alkaline phosphatase kit (BioRad) as per the manufacturer's instructions.

Compound (1), at concentration ranges between 10-20 μM, increased the secretion of soluble APPα (sAPPα) significantly.

### Example 16

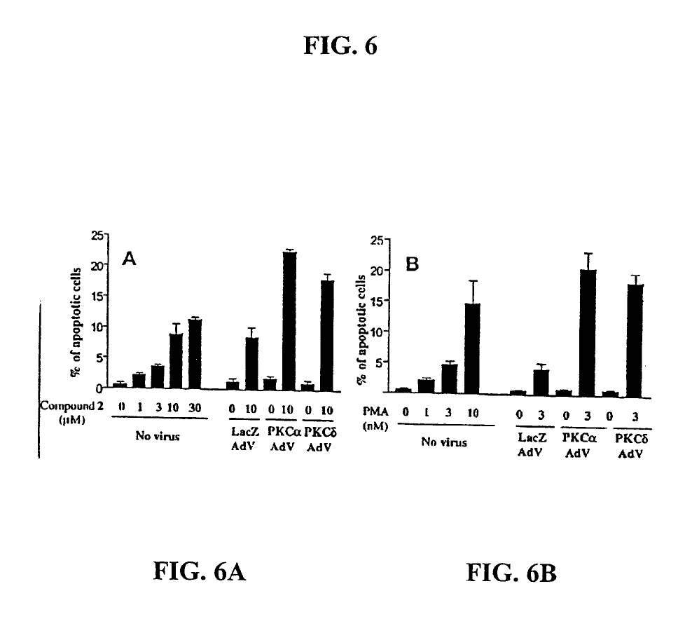

The capacity of inducing apoptosis by compound (1) was evaluated in a model of LNCaP prostate cancer cells. It is well established that in this model phorbol esters (e.g., PMA) activate PKC to induce apoptosis (reference 13). LNCaP cells were treated with compound (1) for 1 h, and apoptosis was assessed 24 h later. As depicted in FIG. 6A, compound (1) (1-30 μM) induces a dose-dependent apoptotic effect in LNCAP cells. For comparison, a representative experiment using the phorbol ester PMA is shown in FIG. 6B.

Control cells or cells infected for 14 h with adenoviruses for LacZ, PKCα, or PKCδ (multiplicity of infection=10 pfu/cell) were treated with different concentrations of compound (1) or PMA for 1 h. Cells were collected 24 h later and stained with DAPI. The incidence of apoptosis in each preparation was analyzed by counting 500 cells and determining the percentage of apoptotic cells. Results are the mean±standard error of three independent experiments.

It is well established that PKCα and PKCδ are the isozymes that mediate phorbol ester-induced apoptosis in LNCaP cells (references 13c-e). A model that has been used to assess isozyme-specificity is the overexpression of specific PKCs by an adenoviral system. Infection of LNCaP cells with adenoviruses for PKCs leads to a high expression of the corresponding PKC isozyme, as determined by Western-blot analysis, kinase activity, or [³H]PDBu binding (reference 13e). Using this system, we found a marked potentation of PMA-induced apoptosis in LNCaP cells by overexpression of PKC isozymes, as also shown in FIG. 6B. Remarkably, a similar effect was observed with compound (1), where overexpression of either PKCα or PKCδ markedly potentiates the apoptotic response in LNCaP prostate cancer cells (FIG. 7A). As expected, a control adenovirus (LacZ adenovirus) did not affect the apoptotic effect of either compound (1) or PMA.

While the compound (1) was found to be less potent than PMA at binding to PKC, it still induces levels of apoptosis in LNCaP cells overexpressing either the PKCα or δ isozymes. As other evidence indicates that lower affinity ligands may prove to be safer when administered systemically, the present results are relevant to drug development focussed on modulation of PKC.

All publications, patents, and patent documents referred to herein are hereby incorporated in their respective entireties by reference. The invention has been described with reference to the foregoing specific and preferred embodiments and methods. However, it should be understood that many variations may be made while remaining within the spirit and scope of the invention. Therefore, the foregoing examples are not limiting, and the scope of the invention is intended to be limited only by the following claims.

What is claimed is:

1. A compound of the general formula 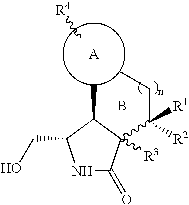
comprising a cyclic substituent A and a substituted cycloalkyl ring B:
wherein R¹, R², and R³, are each independently hydrogen, (C₁-C₁₅)alkyl, (C₂-C₁₅)alkenyl, (C₂-C₁₅)alkynyl, (C₃-C₈)cycloalkyl, (C₃-C₈)cycloalkyl(C₁-C₁₀)alkyl, (C₃-C₈)cycloalkyl(C₁-C₁₅)alkenyl, (C₃-C₈)cycloalkyl(C₁-C₁₅)alkynyl, (C₁-C₁₅)alkoxy, (C₁-C₁₅)alkanoyl, (C₁-C₁₅)alkanoyloxy, aryl heteroaryl, aryl(C₁-C₆)alkyl, heteroaryl(C₁-C₆)alkyl, aryl(C₂-C₁₅)alkenyl, heteroaryl(C₂-C₁₅)alkenyl, aryl(C₂-C₁₅)alkynyl, heteroaryl(C₂-C₁₅)alkynyl, aryl(C₁-C₁₅)alkoxy, heteroaryl(C₁-C₁₅)alkoxy, aryl(C₁-C₁₅)alkanoyl, heteroaryl(C₁-C₁₅)alkanoyl, aryl(C₁-C₁₅)alkanoyloxy, or heteroaryl(C₁-C₁₅)alkanoyloxy; or
wherein R¹ and R² together form a cyclopropyl, cyclobutyl or cyclopentyl ring optionally substituted with one or more substituents R⁵ ring spirocyclic to said substituted cycloalkyl ring B; wherein R⁵ comprises one or more substituents independently selected from the group consisting of hydrogen, (C₁-C₁₅)alkyl, (C₂-C₁₅)alkenyl, (C₂-C₁₅)alkynyl, (C₃-C₈)cycloalkyl, (C₃-C₈)cycloalkyl(C₁-C₁₀)alkyl, (C₃-C₈)cycloalkyl(C₁-C₁₅)alkenyl, (C₃-C₈)cycloalkyl(C₁-C₁₅)alkynyl, (C₁-C₁₅)alkoxy, (C₁-C₁₅)alkanoyl, (C₁-C₁₅)alkanoyloxy, aryl heteroaryl, aryl(C₁-C₆)alkyl, heteroaryl(C₁-C₆)alkyl, aryl(C₂-C₁₅)alkenyl, heteroaryl(C₂-C₁₅)alkenyl, aryl(C₂-C₁₅)alkynyl, heteroaryl(C₂-C₁₅)alkynyl, aryl(C₁-C₁₅)alkoxy, heteroaryl(C₁-C₁₅)alkoxy, aryl(C₁-C₁₅)alkanoyl, heteroaryl(C₁-C₁₅)alkanoyl, aryl(C₁-C₁₅)alkanoyloxy, or heteroaryl(C₁-C₁₅)alkanoyloxy; and R³ is as defined above;
wherein R⁴ comprises 0-4 substituents, independently selected from the group consisting of halo, nitro, cyano, hydroxy, phospho, sulfo, trifluoromethyl, trifluoromethoxy, (C₁-C₁₅)alkyl, (C₂-C₁₅)alkenyl, (C₂-C₁₅)alkynyl, (C₃-C₈)cycloalkyl, (C₃-C₈)cycloalkyl(C₁-C₁₅)alkyl, (C₃-C₈)cycloalkyl(C₂-C₁₅)alkenyl, (C₃-C₈)cycloalkyl(C₂-C₁₅)alkynyl, (C₁-C₁₅)alkoxy, (C₁-C₁₅)alkanoyl, (C₁-C₁₅)alkanoyloxy, C(═O)ORₐ, C(═O)NR_{b}R_{c}, OC(═O)ORₐ, OC(═O)NR_{b}R_{c}, and NR_{d}Rₑ;
wherein each Rₐ is independently hydrogen or (C₁-C₆)alkyl;
wherein each R_{b} and R_{c} is independently hydrogen or (C₁-C₁₀)alkyl; or R_{b}, and R_{c} together with the nitrogen to which they are attached are a 5-6 membered heterocyclic ring; and
wherein each R_{d} and Rₑ is independently hydrogen, (C₁-C₁₀)alkyl, (C₁-C₁₀)alkanoyl, phenyl, benzyl, or phenethyl; or R_{d} and Rₑ together with the nitrogen to which they are attached are a 5-6 membered heterocyclic ring;
wherein n is 0, 1, or 2;
or a pharmaceutically acceptable salt thereof.

2. The compound according to claim 1 of the general formula:

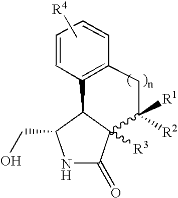

3. A compound according to claim 1 of the general formula: 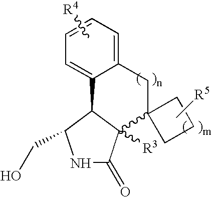
wherein m=0, 1 or 2.

4. The compound of any of claims 1-3, wherein R³ is hydrogen.

5. The compound of claim 1, wherein R² and R³ are in a cis conformation.

6. The compound of one of claims 1-5, wherein n is zero.

7. The compound of one of claims 1-6, wherein R⁴ is (C₂-C₁₅)alkynyl.

8. The compound of one of claims 1, 2 or 4-7, wherein R² is (C₁-C₁₅)alkyl.

9. The compound of one of claims 1-8, wherein R⁴ is 6-(1′-decynyl).

10. The compound of one of claims 1, 2 or 4-9, wherein R² is methyl.

11. The compound of one of claims 1, 2 or 4-9, wherein R² is isopropyl.

12. The compound of claim 5, wherein R¹ is methyl, R²is isopropyl, and R⁴is dodecanoate.

13. A compound as in claim 5, wherein R¹ is isopropyl, R² is methyl, and R⁴ is dodecanoate.

14. A compound as in claim 5, wherein R¹ is isopropyl, R² is methyl, and R⁴ is dodecanoate and acetyl.

15. A compound as in claim 5, wherein R¹ is isopropyl, R² is methyl, and R⁴ is dodecanoate and bromide.

16. A compound as in claim 5, wherein R¹ is isopropyl, R² is methyl, and R⁴ is dodecanoate, bromide, and bromide.

17. A compound as in claim 5, wherein R¹ is isopropyl, R² is methyl, and R⁴ is dodecanoate and 1-heptyne.

18. A compound as in claim 5, wherein R¹ is methyl, R² is isopropyl, and R⁴ is dodecanoate and 1-heptyne.

19. A compound with the formula (3S, 8R, 9S, 10S)-6-(dec-1′-ynyl)-3-hydroxymethyl-8-isopropyl-8-methyl-3,3a,8,8a-tetrahydro-2H-2-aza-cyclopenta[a]inden-1-one: 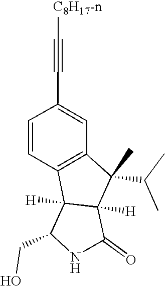
or a pharmaceutically acceptable salt thereof.

20. A compound as in claim 3, wherein m is zero and R consists of 4 methyl groups.

21. A pharmaceutical composition comprising a compound of one of claims 1-20 having a protein kinase C modulating effect in a pharmaceutically acceptable carrier.

22. A composition according to claim 21 wherein the composition is selective for at least one of the classic isozymes of protein kinase C.

23. A composition according to claim 21 wherein the composition is selective for at least one of the novel isozymes of protein kinase C.

24. A composition according to claim 21 wherein the composition is selective for at least one of the atypical isozymes of protein kinase C.

25. A method of modulating protein kinase C activity in a mammal, said method comprising administering to said mammal at least one dose of an efficacious amount of the pharmaceutical composition of claim 21.

26. A method of treating a patient having an autoimmune disease, said method comprising administering to said patient at least one dose of an efficacious amount of the pharmaceutical composition of claim 21.

27. A method of treating a patient having an inflammation, said method comprising administering to said patient at least one dose of an efficacious amount of the pharmaceutical composition of claim 21.

28. A method of treating a patient having a cancer, said method comprising administering to said patient at least one dose of an efficacious amount of the pharmaceutical composition of claim 21.

29. A method of treating prostate cancer in a male patient, said method comprising administering to said male patient at least one dose of an efficacious amount of a pharmaceutical composition of claim 21.

30. A method for reducing β-amyloid accumulation comprising administering an effective amount of the pharmaceutical composition of claim 21.

31. The method of claim 30, wherein said administering an effective amount of said PKC activator reduces plaque formation caused by said β-amyloid accumulation.

32. A method for treating a degenerative neurological disorder comprising administering a rigid pyrrolidone that is a selective PKC activator in an amount effective to achieve a biologically significant (a) increase in soluble α-APP (b) proportional decrease is production of β₁₋₄₀ and β₄₁₋₄₂ relative to α-APP, and/or (c) decrease in β-amyloid aggregation.

33. A method of inhibiting β-amyloid protein accumulation in neurons, said method comprising administering an amount of a rigid pyrrolidone that is a selective PKC activator effective to slow or prevent neurotoxicity.

34. A method of modulating K⁺channel conductance, said method comprising administering an effective amount of a rigid pyrrolidone that is a selective PKC activator.

35. The method of claim 34, wherein the method further comprising altering β amyloid protein accumulation.

36. A method of increasing the amount of sAPP as compared to the amount of β-amyloid protein in CNS neurons, said method comprising administering an effective amount of a rigid pyrrolidone that is a selective PKC activator.

37. A method for increasing the generation of non-amyloidogenic soluble APP comprising activation of protein kinase C (PKC) by administering an effective amount of the composition of claim 21.

38. A method for altering conditions associated with amyloid processing comprising administering an effective amount of a rigid pyrrolidone that is a selective PKC activator effective to enhance an α-secretase pathway to generate soluble α-amyloid precursor protein (α-APP) and prevent β-amyloid aggregation.

39. A composition for treating Alzheimer's disease comprising:
(i) a PKC activator in an amount effective to generate soluble α-APP and prevent β-amyloid aggregation; and
(ii) a pharmaceutically effective carrier wherein said PKC activator is a rigid pyrrolidone.

40. A method for treating plaque formation caused by β-amyloid accumulation comprising administering an effective amount of a rigid pyrrolidone selective PKC activator.

41. A method for treating Alzheimer's disease comprising activation of protein kinase C (PKC) by administering an effective amount of a selective PKC activator.

42. The method of claim 38, wherein said PKC activator is selective for α, β or γ isozymes of PKC.

43. The method of claim 38, wherein said PKC activator is selective for a γ isozyme of PKC.

44. The method of claim 38, wherein said PKC activator is selective for PKC isozymes present in the brain of a subject.

45. The method of claim 41, wherein said PKC isozymes are present in the brain of the subject at concentrations higher than in the remainder of the subject.

46. A method of modulating PKC in a mammalian cell comprising administering to said cell a composition of claim 21.

47. The method of any of claims 25-46 wherein said administering is in vivo or in vitro.

48. The method of any of claims 25-46 wherein said PKC activator is administered to a subject.

49. The method of any of claims 25-46 wherein said PKC activator is administered to a biological sample.

50. The method of any of claims 25-46 wherein said biological sample comprises a cell.

51. The method of any of claims 25-46 wherein said PKC activator is administered together with a pharmaceutically acceptable carrier.
